SEQUENCE LISTING (11 sequences)

SEQ ID NO: 1 - protein, 373 aa, Cricetulus griseus
  REGION 1..373: note = Wild type Cricetulus griseus glutamine synthetase
MATSASSHLNKNIKQMYLCLPQGEKVQAMYIWVDGTGEGLRCKTRTLDCEPKCVEELPEWNFDGSSTFQSEGSNSDMYLSPVAMFRDPFRRDPNKLVFCEVFKYNRKPAETNLRHSCKRIMDMVSNQHPWFGMEQEYTLMGTDGHPFGWPSNGFPGPQGPYYCGVGADKAYGRDIVEAHYRACLYAGVKITGTNAEVMPAQWEFQIGPCEGIRMGDHLWVARFILHRVCEDFGVIATFDPKPIPGNWNGAGCHTNFSTKAMREENGLKHIEEAIEKLSKRHRYHIRAYDPKGGLDNARRLTGFHETSNINDFSAGVANRSASIRIPRTVGQEKKGYFEDRRPSANCDPFAVTEAIVRTCLLNETGDEPFQYKN

SEQ ID NO: 2 - protein, 373 aa, Homo sapiens
  REGION 1..373: note = Wild type Homo sapiens glutamine synthetase
MTTSASSHLNKGIKQVYMSLPQGEKVQAMYIWIDGTGEGLRCKTRTLDSEPKCVEELPEWNFDGSSTLQSEGSNSDMYLVPAAMFRDPFRKDPNKLVLCEVFKYNRRPAETNLRHTCKRIMDMVSNQHPWFGMEQEYTLMGTDGHPFGWPSNGFPGPQGPYYCGVGADRAYGRDIVEAHYRACLYAGVKIAGTNAEVMPAQWEFQIGPCEGISMGDHLWVARFILHRVCEDFGVIATFDPKPIPGNWNGAGCHTNFSTKAMREENGLKYIEEAIEKLSKRHQYHIRAYDPKGGLDNARRLTGFHETSNINDFSAGVANRSASIRIPRTVGQEKKGYFEDRRPSANCDPFSVTEALIRTCLLNETGDEPFQYKN

SEQ ID NO: 3 - protein, 373 aa, Mus musculus
  REGION 1..373: note = Wild type Mus musculus glutamine synthetase
MATSASSHLNKGIKQMYMSLPQGEKVQAMYIWVDGTGEGLRCKTRTLDCEPKCVEELPEWNFDGSSTFQSEGSNSDMYLHPVAMFRDPFRKDPNKLVLCEVFKYNRKPAETNLRHICKRIMDMVSNQHPWFGMEQEYTLMGTDGHPFGWPSNGFPGPQGPYYCGVGADKAYGRDIVEAHYRACLYAGVKITGTNAEVMPAQWEFQIGPCEGIRMGDHLWIARFILHRVCEDFGVIATFDPKPIPGNWNGAGCHTNFSTKAMREENGLKCIEEAIDKLSKRHQYHIRAYDPKGGLDNARRLTGFHETSNINDFSAGVANRGASIRIPRTVGQEKKGYFEDRRPSANCDPYAVTEAIVRTCLLNETGDEPFQYKN

SEQ ID NO: 4 - protein, 373 aa, Rattus norvegicus
  REGION 1..373: note = Wild type Rattus norvegicus glutamine synthetase
MATSASSHLNKGIKQMYMNLPQGEKIQLMYIWVDGTGEGLRCKTRTLDCDPKCVEELPEWNFDGSSTFQSEGSNSDMYLHPVAMFRDPFRRDPNKLVFCEVFKYNRKPAETNLRHSCKRIMDMVSSQHPWFGMEQEYTLMGTDGHPFGWPSNGFPGPQGPYYCGVGADKAYGRDIVEAHYRACLYAGIKITGTNAEVMPAQWEFQIGPCEGIRMGDHLWVARFILHRVCEDFGVIATFDPKPIPGNWNGAGCHTNFSTKAMREENGLRCIEEAIDKLSKRHQYHIRAYDPKGGLDNARRLTGFHETSNINDFSAGVANRSASIRIPRIVGQEKKGYFEDRRPSANCDPYAVTEAIVRTCLLNETGDEPFQYKN

SEQ ID NO: 5 - protein, 373 aa, synthetic construct
  REGION 1..373: note = Cricetulus griseus glutamine synthetase R298K
MATSASSHLNKNIKQMYLCLPQGEKVQAMYIWVDGTGEGLRCKTRTLDCEPKCVEELPEWNFDGSSTFQSEGSNSDMYLSPVAMFRDPFRRDPNKLVFCEVFKYNRKPAETNLRHSCKRIMDMVSNQHPWFGMEQEYTLMGTDGHPFGWPSNGFPGPQGPYYCGVGADKAYGRDIVEAHYRACLYAGVKITGTNAEVMPAQWEFQIGPCEGIRMGDHLWVARFILHRVCEDFGVIATFDPKPIPGNWNGAGCHTNFSTKAMREENGLKHIEEAIEKLSKRHRYHIRAYDPKGGLDNAKRLTGFHETSNINDFSAGVANRSASIRIPRTVGQEKKGYFEDRRPSANCDPFAVTEAIVRTCLLNETGDEPFQYKN

SEQ ID NO: 6 - protein, 373 aa, synthetic construct
  REGION 1..373: note = Cricetulus griseus glutamine synthetase N10S
MATSASSHLSKNIKQMYLCLPQGEKVQAMYIWVDGTGEGLRCKTRTLDCEPKCVEELPEWNFDGSSTFQSEGSNSDMYLSPVAMFRDPFRRDPNKLVFCEVFKYNRKPAETNLRHSCKRIMDMVSNQHPWFGMEQEYTLMGTDGHPFGWPSNGFPGPQGPYYCGVGADKAYGRDIVEAHYRACLYAGVKITGTNAEVMPAQWEFQIGPCEGIRMGDHLWVARFILHRVCEDFGVIATFDPKPIPGNWNGAGCHTNFSTKAMREENGLKHIEEAIEKLSKRHRYHIRAYDPKGGLDNARRLTGFHETSNINDFSAGVANRSASIRIPRTVGQEKKGYFEDRRPSANCDPFAVTEAIVRTCLLNETGDEPFQYKN

SEQ ID NO: 7 - protein, 373 aa, synthetic construct
  REGION 1..373: note = Cricetulus griseus glutamine synthetase N10T
MATSASSHLTKNIKQMYLCLPQGEKVQAMYIWVDGTGEGLRCKTRTLDCEPKCVEELPEWNFDGSSTFQSEGSNSDMYLSPVAMFRDPFRRDPNKLVFCEVFKYNRKPAETNLRHSCKRIMDMVSNQHPWFGMEQEYTLMGTDGHPFGWPSNGFPGPQGPYYCGVGADKAYGRDIVEAHYRACLYAGVKITGTNAEVMPAQWEFQIGPCEGIRMGDHLWVARFILHRVCEDFGVIATFDPKPIPGNWNGAGCHTNFSTKAMREENGLKHIEEAIEKLSKRHRYHIRAYDPKGGLDNARRLTGFHETSNINDFSAGVANRSASIRIPRTVGQEKKGYFEDRRPSANCDPFAVTEAIVRTCLLNETGDEPFQYKN

SEQ ID NO: 8 - protein, 373 aa, synthetic construct
  REGION 1..373: note = Cricetulus griseus glutamine synthetase N10Q
MATSASSHLQKNIKQMYLCLPQGEKVQAMYIWVDGTGEGLRCKTRTLDCEPKCVEELPEWNFDGSSTFQSEGSNSDMYLSPVAMFRDPFRRDPNKLVFCEVFKYNRKPAETNLRHSCKRIMDMVSNQHPWFGMEQEYTLMGTDGHPFGWPSNGFPGPQGPYYCGVGADKAYGRDIVEAHYRACLYAGVKITGTNAEVMPAQWEFQIGPCEGIRMGDHLWVARFILHRVCEDFGVIATFDPKPIPGNWNGAGCHTNFSTKAMREENGLKHIEEAIEKLSKRHRYHIRAYDPKGGLDNARRLTGFHETSNINDFSAGVANRSASIRIPRTVGQEKKGYFEDRRPSANCDPFAVTEAIVRTCLLNETGDEPFQYKN

SEQ ID NO: 9 - protein, 373 aa, synthetic construct
  REGION 1..373: note = Cricetulus griseus glutamine synthetase R298K and N10S
MATSASSHLSKNIKQMYLCLPQGEKVQAMYIWVDGTGEGLRCKTRTLDCEPKCVEELPEWNFDGSSTFQSEGSNSDMYLSPVAMFRDPFRRDPNKLVFCEVFKYNRKPAETNLRHSCKRIMDMVSNQHPWFGMEQEYTLMGTDGHPFGWPSNGFPGPQGPYYCGVGADKAYGRDIVEAHYRACLYAGVKITGTNAEVMPAQWEFQIGPCEGIRMGDHLWVARFILHRVCEDFGVIATFDPKPIPGNWNGAGCHTNFSTKAMREENGLKHIEEAIEKLSKRHRYHIRAYDPKGGLDNAKRLTGFHETSNINDFSAGVANRSASIRIPRTVGQEKKGYFEDRRPSANCDPFAVTEAIVRTCLLNETGDEPFQYKN

SEQ ID NO: 10 - protein, 373 aa, synthetic construct
  REGION 1..373: note = Cricetulus griseus glutamine synthetase R298K and N10T
MATSASSHLTKNIKQMYLCLPQGEKVQAMYIWVDGTGEGLRCKTRTLDCEPKCVEELPEWNFDGSSTFQSEGSNSDMYLSPVAMFRDPFRRDPNKLVFCEVFKYNRKPAETNLRHSCKRIMDMVSNQHPWFGMEQEYTLMGTDGHPFGWPSNGFPGPQGPYYCGVGADKAYGRDIVEAHYRACLYAGVKITGTNAEVMPAQWEFQIGPCEGIRMGDHLWVARFILHRVCEDFGVIATFDPKPIPGNWNGAGCHTNFSTKAMREENGLKHIEEAIEKLSKRHRYHIRAYDPKGGLDNAKRLTGFHETSNINDFSAGVANRSASIRIPRTVGQEKKGYFEDRRPSANCDPFAVTEAIVRTCLLNETGDEPFQYKN

SEQ ID NO: 11 - protein, 373 aa, synthetic construct
  REGION 1..373: note = Cricetulus griseus glutamine synthetase R298K and N10Q
MATSASSHLQKNIKQMYLCLPQGEKVQAMYIWVDGTGEGLRCKTRTLDCEPKCVEELPEWNFDGSSTFQSEGSNSDMYLSPVAMFRDPFRRDPNKLVFCEVFKYNRKPAETNLRHSCKRIMDMVSNQHPWFGMEQEYTLMGTDGHPFGWPSNGFPGPQGPYYCGVGADKAYGRDIVEAHYRACLYAGVKITGTNAEVMPAQWEFQIGPCEGIRMGDHLWVARFILHRVCEDFGVIATFDPKPIPGNWNGAGCHTNFSTKAMREENGLKHIEEAIEKLSKRHRYHIRAYDPKGGLDNAKRLTGFHETSNINDFSAGVANRSASIRIPRTVGQEKKGYFEDRRPSANCDPFAVTEAIVRTCLLNETGDEPFQYKN

(19) United States
(12) Patent Application Publication
(10) Pub. No.: US 2025/0223581 A1
(43) Pub. Date: Jul. 10, 2025

(54) NEW GLUTAMINE SYNTHETASE VARIANTS AS SELECTION MARKER

(71) Applicant: Boehringer Ingelheim International GmbH, Ingelheim am Rhein (DE)

(72) Inventors: Moritz SCHMIDT, Erbach (DE); Simon FISCHER, Staig (DE); Alicia MOLL, Ehingen (DE); Patrick SCHULZ, Warthausen (DE)

(21) Appl. No.: 18/848,533

(22) PCT Filed: Mar. 22, 2023
(86) PCT No.: PCT/EP2023/057313

§ 371 Date: Sep. 19, 2024

(30) Foreign Application Priority Data
Mar. 23, 2022 (EP) 22163850.5

(51) Int. Cl. C12N 9/00; C07K 16/00; C12N 15/85

(52) U.S. Cl. CPC C12N 9/93; C07K 16/00; C12N 15/85; C12Y 603/01002

(57) ABSTRACT

The invention relates to a modified mammalian glutamine synthetase comprising a mutation at amino acid position 10 and/or 298 in a mammalian glutamine synthetase, wherein the mutation is selected from the group consisting of R298K, N10S, N10T and N10Q and to an expression vector, a nucleic acid and a eukaryotic host cell encoding said modified mammalian glutamine synthetase and to its use as a selection marker. The invention further relates to methods for preparing stable cell lines, or for producing a protein of interest using said modified mammalian glutamine synthetase for selection.

### FIELD OF THE INVENTION

The invention relates to a modified mammalian glutamine synthetase comprising a mutation at amino acid position 10 and/or 298 in a mammalian glutamine synthetase, wherein the mutation is selected from the group consisting of R298K, N10S, N10T and N10Q and to an expression vector, a nucleic acid and a eukaryotic host cell encoding said modified mammalian glutamine synthetase and to its use as a selection marker. The invention further relates to methods for preparing stable cell lines, or for producing a protein of interest using said modified mammalian glutamine synthetase for selection.

### BACKGROUND

Chinese hamster ovary (CHO) cells are one of the most commonly employed mammalian cell lines used for the production of therapeutic proteins, such as antibodies. An important aspect is to generate productive and stable cell lines expressing the protein of interest in a short period of time and at high product titers with suitable product quality. To produce a stable and high-producing cell line a selection phase is required to generate stable heterogenous cell pools consisting of different clones which need to be isolated and screened, before the final production cell line can be preserved in a cell bank. Metabolic selection systems such as the dihydrofolate reductase (DHFR) and the glutamine synthetase (GS) selection system are commonly used to improve this process and to generate stable cell lines more efficiently.

In cell lines deficient of the DHFR gene, such as CHO-DG44 cells, selection is performed in the absence of hypoxanthine and thymidine in the medium. Amplification steps by adding increasing concentration of methotrexate (MTX) may be used in addition. The GS selection system is advantageous as it requires fewer gene copies for the survival and hence selection is faster for high producing cell pools.

Glutamine synthetase (EC 6.3.1.2, also known as γ-glutamyl: ammonia ligase) catalyzes ATP-dependent condensation of ammonia and glutamate to form glutamine. Glutamine synthetases are classified in three subgroups: GSI, GSII and GSIII. The CHO GS is a class Il enzyme, the subclass predominantly expressed by eukaryotes, whereas bacterial GS proteins are typically members of the GSI class. While GSI and GSII catalyze the same reaction, they show no or very little sequence similarity except for the residues forming part of the active site and are overall rather different. For example, bacterial type I GS is a 12-subunit complex (Eisenberg et al. (2000), Biochim. Biophys. Acta 1477, 122-145), and GSII has been reported to form two or three pentameric ring stackings (Krajewski et al. (2008), J. Mol. Biol. 375, 217-228). The overall sequence identity between CHO GS and bacterial GS, such as E. coli, is less than 30%.

GS is an ubiquitous enzyme essential for nitrogen metabolism. Thus, GS has been used as selction marker that is introduced via a mammalian expression construct. In cell lines that do not express sufficient levels of endogenous GS, removal of glutamine supplementation from the cell culture media increases the selection pressure on cells. In cell lines having insufficient endogenous GS levels, such as mouse myeloma cell lines, culturing in the absence of glutamine or lack of glutamine supplementation provides sufficient selection pressure to isolate stable recombinant cell lines. In cell lines having sufficient endogenous GS, such as CHO cells, addition of the GS inhibitor methionine sulfoximine (MSX) or generation of GS knockout cells (GS−/− or GS−/+) are required to allow sufficient selection pressure in the absence of glutamine to isolate productive cell lines. Selection stringency in CHO GS knockout cells is strongly improved and expression of the transfected GS gene under the control of a weak promoter has further been reported to improve selection stringency with or without the use of MSX. In addition to selection stringency and productivity, the stability of protein production of high producing clones has been shown to be a critical attribute.

Also, the transfected glutamine synthetase selection marker has substantial influence on the selection process and the phenotypic stability as well as productivity of CHO-based cell lines. For example, the use of attenuated variants of the CHO glutamine synthetase was further shown to improve stability, selection behavior and productivity (Lin et al. (2019), mAbs, 11:5, 965-976; WO2018093331, US20190352631, WO2017197098). Most of the described attenuated variants had mutations in the conserved substrate-binding residues. For instance, two attenuated GS mutants containing R324C and R341C mutations were first identified in two unrelated infants with congenital GS deficiency (Lin et al. (2019), mAbs, 11:5, 965-976). The residues reported to be involved in binding of glutamate are E134, E136, E196, E203, N248, G249, H253, R299, R319, E338 and R340; the residues reported to be involved in binding of ATP are W130, A191, G192, P208, N255, S257, R262, R324 and Y336; and the residues to be involved in binding of ammonia are D63, S66, D162 and E305 (amino acid numbering refers to human GS) (Krajewski et al. (2008), J. Mol. Biol. 375, 217-228 and WO 2018/093331). The respective coordinates and structural factor data are available via the PDB accession codes 2UU7, 20JW and 2QC8. Further, in CN114085819 A residues within 4.5 Å from MSX or ADP/ATP in a predicted complex structure of CHO glutamine synthetase were reported to be identical in human, dog and CHO wild-type glutamine synthetase, namely V197, Q201, Q205, A250, G251, H304, T306 and R324, of which only the single mutant R324C and particularly the triple mutant V197W, A250F and R324K (GSm1) were reported to provide attenuated GS and stable clones.

Although attenuated GS variants harboring mutations in the active site show an increased selection stringency, the duration of the selection process and the overall cell growth characteristics can be impaired, negatively affecting bioprocess performance, making these mutants often not suitable for the manufacturing of therapeutic antibodies. Attenuation of the selection marker is a fine balance between selection stringency, integrated copy numbers and suitable cell culture performance (e.g. growth behavior) to support the manufacturing of recombinant proteins. Thus, there is a need to discover novel attenuated GS variants to improve mammalian expression vectors in cell line development and to generate a panel of selection maker variants with fine-balanced selection stringency, particularly in the absence of GS inhibitors, such as MSX.

### SUMMARY OF THE INVENTION

The present invention demonstrates that three variants of a mammalian glutamine synthetase, harboring a single point mutation, result in the generation of highly productive and stable host cell pools expressing a therapeutic protein, particularly a therapeutic antibody, showing a more stringent selection behavior due to the attenuated activity compared to wildtype glutamine synthetase and beneficial cell culture performance in CHO cells. All three mutations confer an increased selection stringency and unexpectedly resulted in stable host cell pools with higher productivity and/or stability when compared to wildtype GS, making these variants superior in the cell line development. The mutated amino acid positions have not been described yet and are not directly associated with the catalytic binding pocket of the enzyme, but rather seem to influence the stability of the protein by driving the structural interaction. These protein variants are particularly advantageous as they carry mutations outside of the substrate binding site, e.g. at protein interfaces, which are important for the assembly of a multimeric GS protein complex-giving additional means to modulate the attenuation in a more moderate fashion.

More specifically, the present invention relates to a modified mammalian glutamine synthetase comprising a mutation at amino acid position 10 and/or 298 in a mammalian glutamine synthetase having an amino acid sequence of SEQ ID NO: 1, 2, 3 or 4 or an amino acid sequence having at least 95% sequence identity with SEQ ID NO: 1, 2, 3 or 4, wherein the mutation is selected from the group consisting of R298K, N10S, N10T and N10Q. Particularly, the mutation does not interfere with substrate-binding.

In certain embodiments, the modified mammalian glutamine synthetase comprising a mutation selected from the group consisting of R298K, N10S, N10T and N10Q (a) has diminished enzymatic activity compared to the same mammalian glutamine synthetase not comprising the mutation; and/or (b) mediates increased selection stringency and/or genetic stability upon transfection with a polynucleotide encoding the modified mammalian glutamine synthetase and a transgene compared to the same mammalian glutamine synthetase not comprising the mutation.

In another aspect, the invention relates to an expression vector comprising a polynucleotide encoding the modified mammalian glutamine synthetase according to the invention. In certain embodiment, the expression vector further comprises at least one polynucleotide encoding a protein of interest and/or a non-coding RNA, preferably wherein the protein of interest is a therapeutic protein selected from the group consisting of a cytokine, a hormone, a fusion protein, an antibody, an antibody-derived molecule and an antibody mimetic.

In yet another aspect, the invention relates to a nucleic acid sequence comprising a polynucleotide encoding the modified mammalian glutamine synthetase according to the invention operably linked to a eukaryotic promoter, optionally further comprising at least one polynucleotide encoding a protein of interest and/or a non-coding RNA.

In yet another aspect, the invention relates to a eukaryotic host cell comprising a polynucleotide sequence encoding the modified mammalian glutamine synthetase according to the invention, the expression vector according to the invention or the nucleic acid sequence according to the invention.

The eukaryotic host cell is preferably a mammalian host cell, more preferably a rodent cell, even more preferably a CHO cell. In addition or alternatively the eukaryotic host cell is a GS knockout mutant.

The invention further relates to a method for preparing a cell stably expressing a protein of interest and/or a non-coding RNA, comprising (a) introducing the expression vector according to the invention or the nucleic acid according to the invention, optionally further comprising at least one polynucleotide encoding a protein of interest and/or a non-coding RNA, into a eukaryotic host cell, preferably into a mammalian host cell, more preferably a CHO cell; and (b) culturing the eukaryotic host cell in a medium comprising no glutamine under conditions to select for the modified mammalian glutamine synthetase, wherein the optional at least one polynucleotide encoding a protein of interest and/or a non-coding RNA is co-integrated with the polynucleotide encoding the modified mammalian glutamine synthetase into the host cell genome. The method may further comprise a step of a step of isolating a single clone for clonal expansion to prepare a monoclonal cell line.

In yet another aspect, the invention provides a method of producing a protein of interest, comprising (a) introducing the expression vector comprising a polynucleotide encoding a modified mammalian glutamine synthetase according to the invention or the nucleic acid according to the invention, comprising a mutation selected from the group consisting of R298K, N10S, N10T and N10Q and further comprising at least one polynucleotide encoding a protein of interest into a eukaryotic host cell, preferably into a mammalian host cell, more preferably into a CHO cell; (b) culturing the eukaryotic host cell in a medium comprising no glutamine under conditions to select for the modified mammalian glutamine synthetase, wherein the at least one polynucleotide encoding a protein of interest is co-integrated with the polynucleotide encoding the modified mammalian glutamine synthetase into the host cell genome; (c) optionally isolating single clones for clonal expansion to prepare a monoclonal cell line; (d) culturing the eukaryotic host cell under conditions to produce the protein of interest; and (e) harvesting and optionally purifying the protein of interest. The expression vector may be introduced by transfection or transduction.

In yet another aspect, the invention provides a method of producing a protein of interest, comprising (a) providing the eukaryotic host cell according to the invention comprising a polynucleotide encoding a mammalian glutamine synthetase comprising a mutation at amino acid position 10 and/or 298 in a mammalian glutamine synthetase having the amino acid sequence of SEQ ID NO: 1, 2, 3 or 4 or an amino acid sequence having at least 95% sequence identity with SEQ ID NO: 1, 2, 3 or 4, wherein the mutation is selected from the group consisting of R298K, N10S, N10T and N10Q and at least one polynucleotide encoding a protein of interest; (b) culturing the eukaryotic host cell under conditions to produce the protein of interest; and (c) harvesting and optionally purifying the protein of interest. The eukaryotic host cell of the methods of the invention is (a) a mammalian host cell, preferably a rodent cell, more preferably a CHO cell; and/or (b) a GS knockout mutant.

In invention also provides a kit comprising the expression vector according to the invention and a cell culture medium not comprising glutamine.

In yet another aspect, the invention relates to a use of the modified mammalian glutamine synthetase according to the invention as a selection marker for monoclonal eukaryotic cell line generation, preferably a mammalian cell line generation, more rodent cell line generation, even more preferably a CHO cell line generation.

In yet another aspect, the invention relates to a use of the vector according to the invention for producing a protein of interest in a eukaryotic host cell, preferably mammalian host cell, more preferably a rodent cell, even more preferably a CHO cell.

### DESCRIPTION OF THE FIGURES

FIG. 1: CHO-K1-GS cell pools expressing mAb1 and wildtype CHO GS as selection marker. CHO-K1-GS cell pools stably transfected with a mammalian expression vector to express mAb1 and

CHO wildtype GS as a metabolic selection maker (n=5) were cultured in medium not containing L-glutamine. Titer [mg/L] of mAb1 was determined at the indicated days post transfection.

FIG. 2: Viability of CHO-K1-GS cell pools expressing mAb1 and CHO wildtype GS or R298 variants. CHO-K1-GS cell pools stably transfected with an expression cassette to express mAb1 and CHO wildtype GS (wtGS) or one of 6 variants of CHO GS with various mutations at position R298 were cultured in medium not containing L-glutamine. Viability [%] was determined at the indicated days in selection.

FIG. 3: Productivity and mean productivity of CHO-K1-GS cells expressing mAb1 and CHO wildtype GS or CHO GS R298K mutant. CHO-K1-GS cell pools stably transfected with an expression cassette to express mAb1 and CHO wildtype GS or CHO GS R298K mutant cultured in medium not containing L-glutamine. Titer [μg/mL] of mAb1 was determined at the indicated days in culture. A: Productivity of CHO GS R298K mutant and wildtype CHO GS over the course of 20 days. B: Mean productivity of CHO WT GS and CHO GS R298K mutant (** p<0.01).

FIG. 4: Viability of CHO-K1-GS cells expressing mAb1 and CHO wildtype GS or CHO GS N10S, N10Q or N10W mutants. CHO-K1-GS cell pools stably transfected with an expression cassette to express mAb1 and CHO wildtype GS or single point mutants CHO GS N10S, N10Q or N10W were cultured in medium not containing L-glutamine. Viability [%] was determined at the indicated days post transfection.

FIG. 5: Productivity of CHO-K1-GS cells expressing mAb1 and CHO wildtype GS compared to N10S, N10Q or N10W CHO GS variants as selection marker. CHO-K1-GS cell pools stably transfected with an expression cassette to express mAb1 and CHO wildtype GS or CHO GS N10S, N10Q or N10W mutants cultured in medium not containing L-glutamine. Titer [mg/L] of mAb1 was determined at the indicated days once cells reached viabilities>70%.

FIG. 6: Viability of CHO-K1-GS cells expressing mAb1 and CHO wildtype GS or additional CHO GS N10 variants. CHO-K1-GS cell pools stably transfected with an expression cassette to express mAb1 and CHO wildtype GS or single point mutants CHO GS N10S, N10Q, N10W, N10F, N10Y or N10G cultured in medium not containing L-glutamine. CHO-K1-GS cells carrying N10 mutations were found to be phenotypically unstable, except for cells carrying CHO GS N10S or N10Q. Viability [%] was determined at the indicated days post transfection.

FIG. 7: Productivity of CHO-K1-GS cells expressing mAb1 and CHO wildtype GS or CHO GS N10S and R298K. GS CHO comprising two mutations N10S and R298K combined were tested for stable pool generation as well as productivity assessment on clonal level at 384-screening stage. Titer [mg/L] of mAb1 was determined at the end of screening stage.

FIG. 8: Sequence of wild type Cricetulus griseus glutamine synthetase (SEQ ID NO: 1)

FIG. 9: Sequence of wild type Homo sapiens glutamine synthetase (SEQ ID NO: 2)

FIG. 10: Sequence of wild type Mus musculus glutamine synthetase (SEQ ID NO: 3)

FIG. 11: Sequence of wild type Rattus norvegicus glutamine synthetase (SEQ ID NO: 4)

FIG. 12: Sequence of wild type Cricetulus griseus glutamine synthetase R298K (SEQ ID NO: 5)

FIG. 13: Sequence of wild type Cricetulus griseus glutamine synthetase N10S (SEQ ID NO: 6)

FIG. 14: Sequence of wild type Cricetulus griseus glutamine synthetase N10T (SEQ ID NO: 7)

FIG. 15: Sequence of wild type Cricetulus griseus glutamine synthetase N10Q (SEQ ID NO: 8)

FIG. 16: Sequence of wild type Cricetulus griseus glutamine synthetase 298K and N10S (SEQ ID NO: 9)

FIG. 17: Sequence of wild type Cricetulus griseus glutamine synthetase R298K and N10T (SEQ ID NO: 10)

FIG. 18: Sequence of wild type Cricetulus griseus glutamine synthetase R298K and N10Q (SEQ ID NO: 11)

### DETAILED DESCRIPTION

The term “comprises” or “comprising” means “including, but not limited to”. The term is intended to be open-ended, to specify the presence of any stated features, elements, integers, steps or components, but not to preclude the presence or addition of one or more other features, elements, integers, steps, components or groups thereof. The term “comprising” thus includes the more restrictive terms “consisting of” and “essentially consisting of”. With regard to sequences the terms “having an amino acid sequence of” and “comprising an amino acid of” are used interchangeably and include the embodiment “consisting of the amino acid sequence of”. Similarly, the term “encoding” or “encodes” is intended to be open-ended and allows the presence or addition or one or more other features, elements or components. Furthermore, singular and plural forms are not used in a limiting way. As used herein, the singular forms “a”, “an” and “the” designate both the singular and the plural, unless expressly stated to designate the singular only.

The term “protein” is used interchangeably with “amino acid sequence” or “polypeptide” and refers to polymers of amino acids of any length. These terms also include proteins that are post-translationally modified through reactions that include, but are not limited to, glycosylation, acetylation, phosphorylation, glycation or protein processing. Modifications and changes, for example fusions to other proteins, amino acid sequence substitutions, deletions or insertions, can be made in the structure of a polypeptide while the molecule maintains its biological functional activity. For example, certain amino acid sequence substitutions can be made in a polypeptide or its underlying nucleic acid coding sequence and a protein can be obtained with the same properties.

The term “nucleic acid sequence” is used interchangeably with “polynucleotide” and refers to DNA or RNA of any length. In the context of an expression vector, particularly a plasmid and integration into the host cell's genome the person skilled in the art would understand that it refers to a

DNA sequence or molecule.

The term “encodes” and “codes for” refers broadly to any process whereby the information in a polymeric macromolecule is used to direct the production of a second molecule that is different from the first. The second molecule may have a chemical structure that is different from the chemical nature of the first molecule. For example, in some aspects, the term “encode” describes the process of semi-conservative DNA replication, where one strand of a double-stranded DNA molecule is used as a template to encode a newly synthesized complementary sister strand by a DNA-dependent DNA polymerase. In other aspects, a DNA molecule can encode an RNA molecule (e.g., by the process of transcription that uses a DNA-dependent RNA polymerase enzyme). Also, an RNA molecule can encode a polypeptide, as in the process of translation. When used to describe the process of translation, the term “encode” also extends to the triplet codon that encodes an amino acid. In some aspects, an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation.

The term “expression” as used herein refers to transcription and/or translation of a nucleic acid sequence, typically a heterologous nucleic acid sequence, within a host cell. The level of expression of a gene product of interest in a host cell may be determined on the basis of either the amount of corresponding RNA that is present in the cell, or the amount of the polypeptide encoded by the selected sequence. For example, RNA transcribed from a selected sequence can be quantified by Northern blot hybridization, ribonuclease RNA protection, in situ hybridization to cellular RNA or by PCR, such as qPCR. Proteins encoded by a selected sequence can be quantitated by various methods, e.g. by ELISA, by Western blotting, by radioimmunoassay, by immunoprecipitation, by assaying for the biological activity of the protein, by immunostaining of the protein followed by FACS analysis or by homogeneous time-resolved fluorescence (HTRF) assays. The level of expression of a non-coding RNA, such as a miRNA, lncRNA or shRNA may be quantified by PCR, such as qPCR.

The term “stable transfection” as used herein refers to transfection of a polynucleotide comprising integration of the polynucleotide into a host cell genome, as opposed to transiently introduced polynucleotides that remain separate from the genomic DNA of the host cell. The stable integration may occur by homologous recombination or other types of recombination. The stable integration may comprise a step of transient introduction of a heterologous polynucleotide into a host cell. Stable integration of a polynucleotide may also be achieved by transduction using a viral vector, such as a lentivector.

The term “eukaryotic cell” as used herein refers to cells that have a nucleus within a nuclear envelop and include animal cells, human cells, plant cells and yeast cells. In the present invention a “eukaryotic cell” particularly encompasses mammalian cell, such as Chinese hamster ovary (CHO) cell or HEK293 cell derived cells, and yeast cells. Mammalian cells as used herein refer to all cells or cell lines of mammalian origin, such as human or rodent cells. Cells as referred to herein are cells maintained in culture and do not relate to primary cells, but cell lines or cell line derived cells, i.e., to immortalized cells.

The term “mutation” as used herein refers to a substitution of a single amino acid in a nucleic acid sequence. The person skilled in the art will understand that a mutation at a defined amino acid position referred to by the original amino acid (e.g., R298) allows a substitution with any amino acid expect the original amino acid (R298X). The substituting amino acid may further be defined in the one letter code, e.g., R298K.

The term “about” as used herein refers to a variation of 10% of the value specified, for example, about 50% carries a variation from 45 to 55%.

The term “selection stringency” as used herein refers to the duration to reach more than 70% viability and a doubling time of 48h or less of the cell culture following transfection. The longer the time period, the more stringent the selection behavior. Typically, an attenuated glutamine synthetase shows a more stringent selection behavior compared to CHO wildtype glutamine synthetase.

### Modified Mammalian Glutamine Synthetase and its Expression by a Vector or a Eukaryotic Host Cell

The present invention demonstrates that variants of CHO glutamine synthetase, harboring each one or two single amino acid mutation(s) can be applied to generate highly productive and stable CHO pools expressing therapeutic antibodies showing a more stringent selection behavior due an attenuated activity compared to wildtype CHO glutamine synthetase. All mutants confer an increased selection stringency following transfection and unexpectedly resulted in stable cell pools with higher productivity and/or stability when compared to wildtype GS, making these variants superior in the cell line development process. Particularly, these mutants are advantageous in the absence of glutamine synthetase inhibitors, such as MSX.

The present invention provides a modified mammalian glutamine synthetase comprising a mutation at amino acid position 10 and/or 298 in a mammalian glutamine synthetase, wherein the mutation is selected from the group consisting of R298K, N10S, N10T and N10Q and the mammalian glutamine synthetase preferably has the amino acid sequence of hamster glutamine synthase (SEQ ID NO: 1), human glutamine synthetase (SEQ ID NO: 2), mouse glutamine synthetase (SEQ ID NO: 3 or rat glutamine synthetase (SEQ ID NO: 4) or an amino acid sequence having at least 95% sequence identity with SEQ ID NO: 1, 2, 3 or 4.

More specifically in a first aspect the present invention relates to a modified mammalian glutamine synthetase comprising a mutation at amino acid position 10 and/or 298 in a mammalian glutamine synthetase having an amino acid sequence of SEQ ID NO: 1, 2, 3 or 4 or an amino acid sequence having at least 95% sequence identity with SEQ ID NO: 1, 2, 3 or 4, wherein the mutation is selected from the group consisting of R298K, N10S, N10T and N10Q. The mutations in the modified mammalian glutamine synthetase according to the invention does not interfere with substrate binding. Particularly, the mutation is not in a substrate-binding residue. This means the residue has not been (or has not yet been) reported to be involved in binding of glutamate, ATP or ammonia. In certain embodiments the mutation is R298K and/or N10S or N10T or N10Q. Thus, the mutation is R298K or the mutation is N10X, wherein X is S, T or Q (R298K or N10S or N10T or N10Q) or the mutation is R298K and N10X, wherein X is S, T or Q (R298K and N10S or N10T or N10Q). Preferably the mutation N10X is N10S or N10Q. In certain embodiments the mutation is R298K or N10S or N10Q or R298K and N10S or R298K and N10Q.

The mammalian glutamine synthetases according to the invention comprises the mutation in a mammalian glutamine synthetase having at least 95% amino acid sequence identity with SEQ ID NO: 1, 2, 3 or 4. Preferably the glutamine synthetase has at least 96%, at least 97%, at least 98%, at least 99% or 100% sequence identity with SEQ ID NO: 1, 2, 3 or 4. In certain embodiments the glutamine synthetase is a Cricetulus griseus glutamine synthetase, preferably having the amino acid sequence of SEQ ID NO: 1, a human glutamine synthetase, preferably having the amino acid sequence of SEQ ID NO: 2, a mouse glutamine synthetase, preferably having the amino acid sequence of SEQ ID NO: 3 or a rat glutamine synthetase, preferably having the amino acid sequence of SEQ ID NO: 4. In a preferred embodiment the mammalian glutamine synthetase is a Cricetulus griseus glutamine synthetase having at least 95% sequence identity with SEQ ID NO: 1 or at least 96%, at least 97%, at least 98%, at least 99% or 100% sequence identity with SEQ ID NO: 1.

In the context of the present invention, the term “having at least 95% sequence identity with” refers to a protein that comprises a sequence which shares at least 95% of the amino acid residues with a sequence of a reference sequence. Sequence identity can be easily determined by sequence alignment. The sequence may be a natural sequence, such as of a different species as the reference sequence or an allelic variant of the reference sequence or an engineered sequence comprising one or more modifications over the reference sequence.

The modified mammalian glutamine synthetase provided herein comprising a mutation at amino acid position 10 and/or 298 of a mammalian glutamine synthetase having an amino acid sequence of SEQ ID NO: 1, 2, 3, or 4 or an amino acid sequence having at least 95% sequence identity with SEQ ID NO: 1, 2, 3 or 4, wherein the mutation is selected from the group consisting of R298K, N10S, N10T and N10Q may further comprise one or more additional mutations that attenuate glutamine synthase activity or increase catalytic activity to the glutamine synthetase. The further mutations may be a substitution with another amino acid, such as described by Lin et al. (2019) (mAbs, 11:5, 965-976) or in WO2018093331, US20190352631 or WO2017197098.

In certain embodiments the modified glutamine synthetase of the invention, wherein the modified glutamine synthetase comprises a mutation at amino acid position 10 and/or 298 in a mammalian glutamine synthetase, has an amino acid sequence of SEQ ID NO: 5, 6, 7, 8, 9, 10 or 11.

In certain embodiments the modified mammalian glutamine synthetase comprising a mutation selected from the group consisting of R298K, N10S, N10T and N10Q has diminished enzymatic activity compared to the same mammalian glutamine synthetase not comprising the mutation. In an alternative or in an additional embodiment the modified mammalian glutamine synthetase comprising a mutation selected from the group consisting of R298K, N10S, N10T and N10Q mediates increased selection stringency and/or genetic stability upon transfection with a polynucleotide encoding the modified mammalian glutamine synthetase and a transgene compared to the same mammalian glutamine synthetase not comprising the mutation. In yet another embodiments the modified mammalian glutamine synthetase comprising a mutation selected from the group consisting of R298K, N10S, N10T and N10Q has diminished enzymatic activity compared to a mammalian glutamine synthetase having the amino acid sequence of SEQ ID NO: 1, and/or mediates increased selection stringency and/or genetic stability upon transfection with a polypeptide encoding the modified mammalian glutamine synthetase and a transgene compared to a mammalian glutamine synthetase having the amino acid sequence of SEQ ID NO: 1. An increase in selection stringency may be determined by the duration until reaching>70% viability. The transgene may for example encode a protein of interest and/or a non-coding RNA. The term diminished enzyme activity refers to a reduced mammalian glutamine synthetase activity compared to the same mammalian glutamine synthetase without the mutation according to the modified mammalian glutamine synthetase of the invention or compared to the mammalian glutamine synthetase having the amino acid sequence of SEQ ID NO: 1, preferably wherein reduced means reduced by at least 10%, at least 20%, at least 30%, at least 40% or at least 50%. This may be determined, e.g., by the duration of the selection procedure as an indirect read-out.

In a related aspect the invention relates to a use of the modified mammalian glutamine synthetase according to the invention (first aspect) as a selection marker for monoclonal eukaryotic cell line generation, preferably mammalian cell line generation, more preferably a rodent cell line generation, even more preferably a CHO cell line generation.

In a second aspect the invention relates to an expression vector comprising a polynucleotide encoding the modified mammalian glutamine synthetase of the present invention. The embodiments and examples specified with regard to the first aspect similarly apply to this aspect. The expression vector according to the invention is for expression of a heterologous sequence in a eukaryotic host cell, preferably a mammalian host cell, i.e., it is adapted for expression in a eukaryotic or mammalian cell, respectively. Thus, the expression vector according to the invention is characterized in that it comprises a polynucleotide encoding the modified mammalian glutamine synthetase, preferably wherein the polynucleotide encoding the modified mammalian glutamine synthetase is operably linked to a promoter. Typically, the expression vector comprises an expression cassette comprising the polynucleotide encoding the modified mammalian glutamine synthetase is operably linked to a promoter. Preferably the expression vector is a eukaryotic expression vector and the promoter is a eukaryotic promoter, more preferably the expression vector is a mammalian expression vector and the promoter is a mammalian promoter. Mammalian promoters regulate transcription in mammalian cells. Exemplary mammalian promoters, without being limited thereto are simian virus 40 early promoter (SV40), cytomegalovirus immediate-early promoter (CMV), human ubiquitin C promoter (UBC), human elongation factor 1a promoter (EF1A), mouse phosphoglycerate kinase 1 promoter (PGK) and chicken β-actin promoter coupled to CMV early enhancer (CAGG). The expression vector may further comprise bacterial sequences, such as an origin of replication and resistance genes for vector amplification in bacterial cells.

The expression vector typically further comprises at least one polynucleotide encoding a protein of interest and/or a non-coding RNA, preferably an expression cassette comprising at least one polynucleotide encoding a protein of interest and/or a non-coding RNA. The polynucleotide encoding the protein of interest and/or the non-coding RNA is operably linked to a promoter. The protein of interest and/or the non-coding RNA and the modified glutamine synthetase of the present invention may be encoded by the same (multicistronic) or separate expression cassettes. In certain embodiments, the non-coding RNA is an RNA interference (RNAi) mediating RNA, for example miRNA, siRNA, lncRNA or shRNA.

The term “expression cassette” as used herein is a distinct component of a DNA, particularly vector DNA, consisting of one or more coding polynucleotide sequences and the regulatory sequences controlling their expression in a transfected or transduced cell. An expression cassette comprises at least three components: a promoter sequence, an open reading frame, and termination sequence. In eukaryotic expression vectors comprising a polynucleotide encoding a protein of interest, the termination sequence is referred to as 3′ untranslated region and usually contains a polyadenylation site. The expression cassette directs the cell's machinery to make RNA and may therefore also be referred to as transcriptional cassette. The RNA may be coding RNA, further processed to mRNA encoding for a protein sequence e.g., glutamine synthetase or the protein of interest, or the RNA may be non-coding RNA, such as RNA interference (RNAi) mediating RNA, for example miRNA, siRNA, lncRNA or shRNA.

The protein of interest may be any protein, but is typically a therapeutic protein. These include, but are not limited to cytokines, growth factors, hormones, blood coagulation factors, vaccines, interferons, fusion proteins, antibodies, antibody-derived molecules and an antibody mimetics. In certain embodiments, the therapeutic protein is selected from the group consisting of a cytokine, a hormone, a fusion protein, an antibody, an antibody-derived molecule and an antibody mimetic.

In certain embodiments the protein of interest is an antibody. In cases where the protein of interest is an antibody, the eukaryotic expression vector (particularly the mammalian expression vector) comprises a polynucleotide comprising a coding sequence for a variable region of the heavy chain and/or a coding sequence for a variable region of the light chain of the antibody. In certain embodiments, the eukaryotic expression vector comprises a polynucleotide comprising a coding sequence for a heavy chain and/or a coding sequence for a light chain of the antibody. Thus, the polynucleotide comprising a coding sequence for a variable region of the heavy chain and the polynucleotide comprising a coding sequence of a variable region of the light chain may be expressed on the same eukaryotic expression vector or on separate eukaryotic expression vectors. The expression vector may comprise a multicistronic expression cassette, such as a bicistronic expression cassette, and/or multiple expression cassettes. A multicistronic expression cassette comprises more than one open reading frames separated by sequences coding for an RNA element that allows for translation initiation, such as an internal ribosomal entry site (IRES). In a multicistonic expression cassette, the two or more open reading frames are under the control of the same promoter. The polynucleotide encoding at least a variable region of the heavy chain and/or the polynucleotide encoding at least a variable region of the light chain may therefore be expressed within the same expression cassette (separated e.g., by an IRES sequence) or by two separate expression cassettes. Moreover, the bacterial glutamine synthetase and the protein of interest and/or the non-coding RNA may be expressed by the same or separate expression cassette(s). In case the protein of interest is an antibody, the bacterial glutamine synthetase and polynucleotide encoding at least a variable region of the heavy chain and/or the polynucleotide encoding at least a variable region of the light chain may be expressed by the same or separate expression cassettes or a mixture thereof.

In preferred embodiments, the eukaryotic expression vector is for stable integration into the host cell's genome (such as for stable transfection) and the integrating part of the vector comprises the polynucleotide encoding the bacterial glutamine synthetase and the at least one polynucleotide encoding the protein of interest and/or the non-coding RNA. The eukaryotic expression vector according to the invention is a plasmid, a Bacterial Artificial Chromosome (BAC) or a viral vector. Said plasmid, BAC or viral vector (e.g. a lentiviral vector) may be introduced into the eukaryotic host cell (such as a mammalian host cell) via transfection or transduction, respectively, and is preferably stably integrated into the host cell genome. The person skilled in the art knows suitable plasmids BACs or viral vectors and that a plasmid may further comprise transposon recognition sequences upstream and downstream of the polynucleotide encoding a bacterial synthetase as a selection marker and the optional at least one polynucleotide encoding a protein of interest and/or a non-coding RNA.

A preferred protein of interest is an antibody, including fragments and derivatives thereof. Typically, an antibody is monospecific, but an antibody may also be multispecific. Thus, the present invention may be used for the production of mono-specific antibodies, multi-specific antibodies, or fragments thereof, preferably of antibodies (mono-specific), bispecific antibodies, trispecific antibodies or fragments thereof, preferably antigen-binding fragments thereof. Exemplary antibodies within the scope of the present invention include but are not limited to anti-CD2, anti-CD3, anti-CD20, anti-CD22, anti-CD30, anti-CD33, anti-CD37, anti-CD40, anti-CD44, anti-CD44v6, anti-CD49d, anti-CD52, anti-EGFR1 (HER1), anti-EGFR2 (HER2), anti-GD3, anti-IGF, anti-VEGF, anti-TNFalpha, anti-IL2, anti-IL-5R or anti-IgE antibodies, and are preferably selected from the group consisting of anti-CD20, anti-CD33, anti-CD37, anti-CD40, anti-CD44, anti-CD52, anti-HER2/neu (erbB2), anti-EGFR, anti-IGF, anti-VEGF, anti-TNFalpha, anti-IL2 and anti-IgE antibodies.

The term “antibody”, “antibodies”, or “immunoglobulin(s)” is used herein in the broadest sense and encompasses various antibody structures, including but not limited to monoclonal antibodies, polyclonal antibodies, monospecific antibodies, multispecific antibodies (e.g., bispecific antibodies), and antibody fragments so long as they exhibit the desired antigen-binding activity. There are various classes of immunoglobulins: IgA, IgD, IgE, IgG, IgM, IgY, IgW. Preferably the antibody is an lgG antibody, more preferably an IgG1 or an lgG4 antibody.

Antibodies can be of any species and include chimeric and humanized antibodies. “Chimeric” antibodies are molecules in which antibody domains or regions are derived from different species. For example, the variable region of heavy and light chain can be derived from rat or mouse antibody and the constant regions from a human antibody. In “humanized” antibodies only minimal sequences are derived from a non-human species. Often only the CDR amino acid residues of a human antibody are replaced with the CDR amino acid residues of a non-human species such as mouse, rat, rabbit or llama. Sometimes a few key framework amino acid residues with impact on antigen binding specificity and affinity are also replaced by non-human amino acid residues.

Typically, antibodies are tetrameric polypeptides composed of two pairs of a heterodimer each formed by a heavy and a light chain. Stabilization of both the heterodimers as well as the tetrameric polypeptide structure occurs via interchain disulfide bridges. Each chain is composed of structural domains called “immunoglobulin domains” or “immunoglobulin regions” whereby the terms “domain” or “region” are used interchangeably. Each domain contains about 70-110 amino acids and forms a compact three-dimensional structure. Both heavy and light chain contain at their N-terminal end a “variable domain” or “variable region” with less conserved sequences which is responsible for antigen recognition and binding. The variable region of the light chain is also referred to as “VL” and the variable region of the heavy chain as “VH”.

An “antibody fragment” or “antigen-binding fragments” refers to a molecule other than an intact antibody that comprises a portion of an intact antibody that binds the antigen to which the intact antibody binds. Examples of antibody fragments include but are not limited to Fv, Fab, Fab′, Fab′-SH,

F(ab′) 2; diabodies; linear antibodies; single-chain antibody molecules (e.g. scFv); and multispecific antibodies formed from antibody fragments. Fab fragments consist of the variable regions of both chains, which are held together by the adjacent constant region. These may be formed by protease digestion, e.g. with papain, from conventional antibodies, but similarly Fab fragments may also be produced by genetic engineering. Further antibody fragments include F(ab′) 2 fragments, which may be prepared by proteolytic cleavage with pepsin.

Using genetic engineering methods it is possible to produce shortened antibody fragments which consist only of the variable regions of the heavy (VH) and of the light chain (VL). These are referred to as Fv fragments (Fragment variable=fragment of the variable part). Since these Fv-fragments lack the covalent bonding of the two chains by the cysteines of the constant chains, the Fv fragments are often stabilized. It is advantageous to link the variable regions of the heavy and of the light chain by a short peptide fragment, e.g. of 10 to 30 amino acids, preferably 15 amino acids. In this way a single peptide strand is obtained consisting of VH and VL, linked by a peptide linker. An antibody protein of this kind is known as a single-chain-Fv (scFv). Examples of scFv-antibody proteins are known to the person skilled in the art. Thus, antibody fragments and antigen-binding fragments further include Fv-fragments and particularly scFv.

In recent years, various strategies have been developed for preparing scFv as a multimeric derivative. This is intended to lead, in particular, to recombinant antibodies with improved pharmacokinetic and biodistribution properties as well as with increased binding avidity. In order to achieve multimerisation of the scFv, scFv were prepared as fusion proteins with multimerisation domains. The multimerisation domains may be, e.g. the CH3 region of an IgG or coiled coil structure (helix structures) such as Leucine-zipper domains. However, there are also strategies in which the interaction between the VH/VL regions of the scFv is used for the multimerisation (e.g. dia-, tri- and pentabodies). By diabody the skilled person means a bivalent homodimeric scFv derivative. The shortening of the linker in a scFv molecule to 5-10 amino acids leads to the formation of homodimers in which an inter-chain VH/VL-superimposition takes place. Diabodies may additionally be stabilized by the incorporation of disulfide bridges. Examples of diabody-antibody proteins are known from the prior art.

By minibody the skilled person means a bivalent, homodimeric scFv derivative. It consists of a fusion protein which contains the CH3 region of an immunoglobulin, preferably lgG, most preferably lgG1 as the dimerisation region which is connected to the scFv via a Hinge region (e.g. also from lgG1) and a linker region. Examples of minibody-antibody proteins are known from the prior art.

By triabody the skilled person means a: trivalent homotrimeric scFv derivative. ScFv derivatives wherein VH-VL is fused directly without a linker sequence lead to the formation of trimers.

The skilled person will also be familiar with so-called miniantibodies which have a bi-, tri- or tetravalent structure and are derived from scFv. The multimerisation is carried out by di-, tri- or tetrameric coiled coil structures. In a preferred embodiment of the present invention, the gene of interest is encoded for any of those desired polypeptides mentioned above, preferably for a monoclonal antibody, a derivative or fragment thereof.

Further encompassed is a single-domain antibody (sdAb), also be referred to as nanobody, which is an antibody fragment of a single monomeric variable antibody domain. Single-domain antibodies are typically engineered from heavy chain antibodies found in camelids (VHH fragments) or cartilaginous fishes (VNAR fragments).

The immunoglobulin fragments composed of the CH2 and CH3 domains of the antibody heavy chain are called “Fc fragments”, “Fc region” or “Fc” because of their crystallization propensity (Fc=fragment crystallizable). These may be formed by protease digestion, e.g. with papain or pepsin from conventional antibodies but may also be produced by genetic engineering. The N-terminal part of the Fc fragment might vary depending on how many amino acids of the hinge region are still present.

Antibodies comprising an antigen-binding fragment and an Fc region may also be referred to as full-length antibody. Full-length antibody may be mono-specific and multispecific antibodies. Multispecific antibodies are antibodies which have at least two different antigen-binding sites each of which bind to different epitopes. A multispecific antibody includes bispecific and trispecific antibodies.

A bispecific antibody has two different binding binding sites. Multispecific antibodies also include antibody formats other than full-length antibodies such as antibody-derived molecules.

Bispecific antibodies typically combine antigen-binding specificities for target cells (e.g., malignant B cells) and effector cells (e.g., T cells, NK cells or macrophages) in one molecule. Exemplary bispecific antibodies, without being limited thereto are diabodies, BiTE (Bi-specific T-cell Engager) formats and DART (Dual-Affinity Re-Targeting) formats. The diabody format separates cognate variable domains of heavy and light chains of the two antigen binding specificities on two separate polypeptide chains, with the two polypeptide chains being associated non-covalently. The DART format is based on the diabody format, but it provides additional stabilization through a C-terminal disulfide bridge. Trispecific antibodies are monoclonal antibodies which combine three antigen-binding specificities. They may be build on bispecific-antibody technology that reconfigures the antigen-recognition domain of two different antibodies into one bispecific molecule. For example, trispecific antibodies have been generated that target CD38 on cancer cells and CD3 and CD28 on T cells. Multispecific antibodies are particularly difficult to product with high product quality.

The term “antibody-derived molecule” as used herein refers to any molecule comprising at least an antigen-binding moiety that is structurally related to antibodies. It includes modified full-length mono- or bispecific antibodies further modified with an additional antigen binding moiety or smaller antibody formats including the ones described herein.

The term “antibody mimetic” as used herein refers to proteins that bind to specific antigens in a manner similar to antibodies, but that are not structurally related to antibodies. Antibody mimetic include, without being limited thereto an anticalin, an affibody, an adnectin, a monobody, a DARPin, an affimer, and an affitin.

A single-domain antibody (sdAb) may also be referred to as nanobody. The person skilled in the art will understand that the protein may comprise more than one antigen-binding domain and hence may be multivalent, preferably bivalent (e.g., a bivalent sdAb or a bivalent anticalin or any other bivalent antibody mimetic).

Another preferred therapeutic protein is a fusion protein, such as an Fc-fusion protein. Thus, the invention can be advantageously used for production of fusion proteins, such as Fc-fusion proteins. The effector part of the fusion protein can be the complete sequence or any part of the sequence of a natural or modified heterologous protein. The immunoglobulin constant domain sequences may be obtained from any immunoglobulin subtypes, such as lgG1, lgG2, lgG3, lgG4, IgA1 or IgA2 subtypes or classes such as IgA, IgE, IgD or IgM. Preferentially they are derived from human immunoglobulin, more preferred from human IgG and even more preferred from human lgG1 and lgG2. Non-limiting examples of Fc-fusion proteins are MCP1-Fc, ICAM-Fc, EPO-Fc and scFv fragments or the like coupled to the CH2 domain of the heavy chain immunoglobulin constant region comprising the N-linked glycosylation site. Fc-fusion proteins can be constructed by genetic engineering approaches by introducing the CH2 domain of the heavy chain immunoglobulin constant region comprising the N-linked glycosylation site into another expression construct comprising for example other immunoglobulin domains, enzymatically active protein portions, or effector domains. Thus, an Fc-fusion protein according to the present invention comprises also a single chain Fv fragment linked to the CH2 domain of the heavy chain immunoglobulin constant region comprising, e.g., the N-linked glycosylation site.

The term “cytokine” refers to small proteins, which are released by cells and act as intercellular mediators, for example influencing the behavior of the cells surrounding the secreting cell. Cytokines may be secreted by immune cells or other cells, such as T-cells, B-cells, NK cells and macrophages. Cytokines may be involved in intercellular signaling events, such as autocrine signaling, paracrine signaling and endocrine signaling. They may mediate a range of biological processes including, but not limited to immunity, inflammation, and hematopoiesis. Cytokines may be chemokines, interferons, interleukins, lymphokines or tumor necrosis factors.

As used herein, “growth factor” refers to proteins or polypeptides that are capable of stimulating cell growth. They include, but are not limited to, insulin, epidermal growth factor (EGF), ephrins (Eph), Erythropoietin, glia-cell stimulating factor (GSF); colony-stimulating factors (CSF) including macrophage colony-stimulating factor (M-CSF), granulocyte macrophage colony-stimulating factor (GM-CSF), and granulocyte colony-stimulating factor (G-CSF); stem cell growth factor (SCGF) (also called Steel Factor); stromal cell-derived factor (SDF), effective fragments thereof, and combinations thereof; and vascular endothelial growth factor (VEGF). Other growth factors can include hepatocyte growth factor (HGF), Angiopoietin-1, Angiopoietin-2, b-FGF, and FLT-3 ligand, and effective fragment thereof.

In a third aspect, the invention relates to a kit comprising the expression vector according to the invention and optionally a cell culture medium not comprising glutamine.

In a fourth aspect the invention further relates to a use of the expression vector of the invention (second aspect) for expression of a protein of interest and/or a non-coding RNA in a eukaryotic host cell, particularly a mammalian host cell, more preferably a rodent cell, such as a CHO cell. The modified mammalian glutamine synthetase encoded by said expression vector serves as a selection marker in said host cells. In certain embodiments, the use of the expression vector of the invention is for producing a protein of interest in a in a eukaryotic host cell, particularly mammalian, such as a rodent cell, such as a CHO cell. The embodiments and examples specified with regard to the second and first aspect similarly apply to this aspect.

In a fifth aspect the invention relates to a nucleic acid sequence comprising a polynucleotide encoding the modified mammalian glutamine synthetase of the present invention operably linked to a promoter, preferably a eukaryotic promoter (such as a mammalian promoter), optionally further comprising at least one polynucleotide encoding a protein of interest and/or a non-coding RNA. Specifically, the encoded modified mammalian glutamine synthetase is the modified mammalian glutamine synthetase of the first aspect. The nucleic acid sequence may be part of the expression vector of the second aspect. Thus, the embodiments and examples specified with regard to the first and second aspect similarly apply to this aspect. In particular, in certain embodiment the nucleic acid comprises modified mammalian glutamine synthetase comprising a mutation at amino acid position 10 and/or 298 in a mammalian glutamine synthetase having an amino acid sequence of SEQ ID NO:
1, 2, 3 or 4 or an amino acid sequence having at least 95% sequence identity with SEQ ID NO: 1, 2, 3 or 4, wherein the mutation is selected from the group consisting of R298K, N10S, N10T and N10Q, preferably the mutation is R298K and/or N10S/Q.

In a sixth aspect, the invention relates to a eukaryotic host cell comprising a polynucleotide sequence encoding the modified mammalian glutamine synthetase of the present invention (first aspect), the expression vector of the present invention (second aspect) or the nucleic acid sequence of the present invention (fifth aspect). In certain embodiments the eukaryotic host cell comprises the polynucleotide encoding the modified mammalian glutamine synthetase co-integrated with the at least one polynucleotide encoding a protein of interest and/or a non-coding RNA into the host cell genome.

The eukaryotic host cell may be any host cell, provided the host cell is an immortalized cell and not a primary cell. Preferably the eukaryotic host cell is a mammalian host cell or a yeast host cell, more preferably a mammalian host cell. In certain embodiments the mammalian host cell is a mouse, a human or a rodent cell, more preferably a rodent cell, even more preferably a CHO cell. Moreover, the eukaryotic host cell is preferably a GS gene knockout cell (GS knockout mutant) host cell, such as a mammalian GS gene knockout cell. The term “GS gene knockout cell” as used herein refers to a cell in which the endogenous GS gene has been knocked out, i.e., deleted or disrupted, resulting in GS enzyme function disruption. Such cells may be referred to as GS−/− or GS−/+ cells, depending on whether both or only one allele has been deleted or disrupted. Extracellular glutamine supplementation or a GS gene introduced by an expression vector is essential for cell survival of GS gene knockout cells. In a preferred embodiment the mammalian host cell is a CHO-K1 cell, more preferably a CHO-K1-GS (GS−/−) cell. In certain embodiments the eukaryotic or mammalian host cell is a monoclonal cell line generated by a step of single cell cloning and clonal expansion.

### Method for Preparing a Cell Line or Producing a Protein

In an seventh aspect, the invention relates to a method for preparing a cell stably expressing a protein of interest and/or a non-coding RNA, comprising (a) introducing the expression vector according to the invention (second aspect) or the nucleic acid of the invention (fifth aspect) comprising the modified mammalian glutamine synthetase of the present invention (first aspect), optionally further comprising at least one polynucleotide encoding a protein of interest and/or a non-coding RNA, into a eukaryotic host cell, preferably into a CHO cell; and (b) culturing the eukaryotic host cell in a medium comprising no glutamine under conditions to select for the modified mammalian glutamine synthetase, wherein the optional at least one polynucleotide encoding a protein of interest and/or a non-coding RNA is co-integrated with the polynucleotide encoding the modified mammalian glutamine synthetase into the host cell genome. Optionally the method may further comprise a step of culturing the resulting cell pool stably expressing a protein of interest and/or a non-coding RNA selected in step (b) and/or isolating or culturing a cell pool stably expressing a protein of interest and/or a non-coding RNA. A cell stably expressing a protein of interest and/or a non-coding RNA means that the polynucleotide encoding the protein or interest and/or the non-coding RNA is stable integrated into the genome of the host cell and that the protein of interest and/or the non-coding RNA is stable expressed, i.e., over an extended period of time, such as at least 40 days preferably for months or years.

The expression vector or the nucleic acid may be a plasmid, Bacterial Artificial Chromosome (BAC) or a viral vector. Thus, the expression vector or the nucleic acid of the invention may be introduced by transfection or transduction, respectively. Preferably the expression vector or the nucleic acid is a plasmid. More preferably the expression vector or the nucleic acid is introduced by stable transfection. Methods of transducing or transfecting an expression vector or a nucleic acid into eukaryotic cells are well known in the art and comprise chemical means, such as calcium phosphate precipitation and lipofection, and physical means, such as electroporation. The polynucleotide encoding the modified mammalian glutamine synthetase and the polynucleotide encoding the protein of interest and/or the non-coding RNA are operably linked to a eukaryotic promoter and are therefore adapted for expression in a eukaryotic host cell.

The method of the invention may further comprise (c) a step of isolating a single clone for clonal expansion to prepare a monoclonal cell line. The person skilled in the art would understand that transfection or transduction often requires a large number of cells, resulting in a heterogenous pool of recombinant cells with, e.g., varying integration site populations. For generating a clonal cell line, the cell pool is diluted or sorted for single cell isolation (monoclonal) and each single clone is subjected to clonal expansion to prepare a monoclonal cell line. The term “cell line” as used herein refers to a population of cell derived from a single cell clone and can be grown for an unlimited time. It is therefore also referred to as monoclonal cell line. Thus, a cell line is genetically stable and hence the characteristics of a cell line should not change over time. Particularly phenotypic characteristics such as production levels (titer) and growth rate and density (VCD and maximal VCD) viability as well as genetic integrity as measured via copy number and DNA-fingerprint assays should be maintained when cultured under comparable conditions.

The cell pool or the monoclonal cell line prepared according to the method of the invention may be further used for stably producing a protein of interest or for stably producing a non-coding RNA, such as an RNA mediating RNAi, e.g., an miRNA, an siRNA, lncRNA or an shRNA. RNAi is used for gene silencing and may therefore be used for generating a cell pool or a monoclonal cell line in beneficial properties for, e.g., protein production. For example, a difficult to remove host cell protein (HCP) may be silenced in the cell pool or monoclonal cell line, or an enzyme such as a fucosyltransferase may be silenced to modify the glycosylation profile of the cell pool or monoclonal cell line. Moreover, CHO cells commonly used for large-scale industrial production are often engineered to improve their characteristics in the production process, or to facilitate selection of recombinant cells. Such engineering includes, but is not limited to increasing apoptosis resistance, reducing autophagy, increasing cell proliferation, altered expression of cell-cycle regulating proteins, chaperone engineering, engineering of the unfolded protein response (UPR), engineering of secretion pathways and metabolic engineering. Such engineering can potentially be achieved using RNAi in eukaryotic host cells generated by the methods of the present invention.

In a related eighth aspect the invention relates to a method of producing a protein of interest, comprising (a) introducing the expression vector comprising a polynucleotide encoding a modified mammalian glutamine synthetase according to the invention (second aspect) or the nucleic acid of the invention (fifth aspect), comprising a mutation selected from the group consisting of R298K, N10S, N10T and N10Q and further comprising at least one polynucleotide encoding a protein of interest into a eukaryotic host cell; (b) culturing the eukaryotic host cell in a medium comprising no glutamine under conditions to select for the modified mammalian glutamine synthetase, wherein the at least one polynucleotide encoding a protein of interest is co-integrated with the polynucleotide encoding the modified mammalian glutamine synthetase into the host cell genome; (c) optionally isolating a single clone for clonal expansion to prepare a monoclonal cell line; (d) culturing the eukaryotic host cell under conditions to produce the protein of interest; and (e) harvesting and optionally purifying the protein of interest. The expression vector or the nucleic acid of the invention may be introduced by transfection or transduction. Preferably the expression vector or the nucleic acid is introduced by stable transfection.

The person skilled in the art would understand that for an expression vector typically only a part of the vector is integrated into the host cell's genome. Thus, the integrating part of the vector comprises the polynucleotide encoding the modified mammalian glutamine synthetase and at least one polynucleotide encoding the protein of interest and/or the non-coding RNA. Moreover, the integrated part of the expression vector or the integrated nucleic acid sequence of the invention may further be amplified, e.g., by increasing the concentration of a glutamine synthetase inhibitor, such as methionine sulfoximine (MSX). Amplification is optional and may result in higher productivity due to higher copy numbers of the polynucleotide encoding the protein of interest and/or the non-coding RNA, because these become co-amplified together with the modified mammalian glutamine synthetase. The method according to the invention may include the generation of a cell pool or a monoclonal cell line. Further, a eukaryotic host cell generated according to the method of the invention or a eukaryotic host cell according to the invention may further be used for producing a protein of interest and/or a non-coding RNA or in a method for producing a protein of interest.

Thus, in a further nineth aspect, the invention relates to a method of producing a protein of interest, comprising (a) providing the eukaryotic host cell of the invention (sixth aspect) comprising a polynucleotide encoding a modified mammalian glutamine synthetase comprising a mutation at amino acid position 10 and/or 298 in a mammalian glutamine synthetase having the amino acid sequence of SEQ ID NO: 1, 2, 3 or 4 or an amino acid sequence having at least 95% sequence identity with SEQ ID NO: 1, 2, 3 or 4, wherein the mutation is selected from the group consisting of R298K, N10S, N10T and N10Q and at least one polynucleotide encoding a protein of interest; (b) culturing the eukaryotic host cell under conditions to produce the protein of interest; and (c) harvesting and optionally purifying the protein of interest.

In a preferred embodiment of the methods of the invention, the eukaryotic host cell is a GS gene knockout cell. The person skilled in the art will understand that this refers to the endogenous GS gene, while the modified mammalian GS gene is present in the host cell following transfection or transduction of the expression vector or the nucleic acid sequence of the invention. The eukaryotic host cell (transfected or transduced with the expression vector or the nucleic acid sequence of the invention) is cultured in a medium comprising no glutamine under conditions to select for the modified mammalian glutamine synthetase in step (b). This may further comprise the addition of the GS inhibitor methionine sulfoximine (MSX).

In certain embodiments of the methods of the invention the cell (cell pool) or monoclonal cell line is generated with increased selection stringency and/or has increased genetic stability and/or has higher productivity compared to a cell or monoclonal cell line generated with the same mammalian glutamine synthetase not comprising the at least one mutation and/or compared to a cell or monoclonal cell line generated with a mammalian glutamine synthetase having the amino acid sequence of SEQ ID NO: 1. Genetic stability may be quantified by measuring by measuring copy numbers of the integrated transgenes. Genetic stability is assessed by measuring copy number over an extended cultivation time. In addition, genomic rearrangements are monitored via e.g. Southern Blot analysis. An increase in selection stringency may be determined by the duration until reaching>70% viability.

The eukaryotic host cell may be any host cell, provided the host cell is an immortalized cell and not a primary cell. The methods described herein are in vitro methods and the eukaryotic host cells according to the invention are for in vitro use in cell culture. Eukaryotic host cells encompass particularly yeast cells and mammalian cells and are preferably mammalian cells. Yeast cells can be, without being limited thereto Saccharomyces cerevisiae, Pichia pastoris, Klyveromyces lactis or marxianus. The term “mammalian cell” as used herein refers to mammalian cell lines suitable for the production of a product of interest, such as a heterologous protein of interest and/or a non-coding RNA and may also be referred to as “host cells” or “mammalian host cell”. The mammalian cells are preferably transformed and/or immortalized cell lines. They are adapted to serial passages in cell culture, preferably serum-free cell culture and/or preferably as suspension culture, and do not include primary non-transformed cells or cells that are part of an organ structure.

Preferably the mammalian host cell is a mouse, a human or rodent cell, more preferably a rodent cell, even more preferably a CHO cell. Preferred mammalian cells for heterologous protein production are murine cells, rodent cells or human cells. Preferred examples of mammalian cells or mammalian cell lines are CHO cells (such as DG44 and K1), NSO cells, HEK293 cells (such as HEK293 cells and HEK293T cells) and BHK21 cells. Preferably the mammalian cells or mammalian cell lines are adapted to growth in suspension. In a preferred embodiment the mammalian cells or mammalian cell line is a CHO cell. In certain embodiments the mammalian cell is a HEK293 cell or a CHO cell or a HEK293 cell or a CHO cell derived cell, preferably the mammalian cell is a CHO cell or a CHO derived cell.

Suitable rodent cells may be e.g., hamster cells, particularly BHK21, BHK TK-, CHO, CHO-K1, CHO-DXB11 (also referred to as CHO-DUKX or DuxB11), a CHO-S cell and CHO-DG44 cells or the derivatives/progenies of any of such cell line. Particularly preferred are CHO cells, such as CHO-DG44, CHO-K1 and BHK21, and even more preferred are CHO-DG44 and CHO-K1 cells. Most preferred are CHO-DG44 cells. Glutamine synthetase (GS)-deficient derivatives of the mammalian cell, particularly of the CHO-DG44 and CHO-K1 cell are also encompassed. In one embodiment of the invention the mammalian cell is a Chinese hamster ovary (CHO) cell, preferably a CHO-DG44 cell, a CHO-K1 cell, a CHO DXB11 cell, a CHO-S cell, a CHO GS deficient cell or a derivative thereof. Suitable human cells are HEK293 or HEK293T cells. The host cells may also be murine cells such as murine myeloma cells, such as NSO and Sp2/0 cells or the derivatives/progenies of any of such cell line.

Moreover, the eukaryotic host cell is preferably a GS gene knockout cell (GS knockout mutant) host cell. The term “GS gene knockout cell” as used herein refers to a cell in which the endogenous GS gene has been knocked out, i.e., deleted or disrupted, resulting in GS enzyme function disruption.

Such cells may be referred to as GS−/− or GS−/+ cells, depending on whether both or only one allele has been deleted or disrupted. Extracellular glutamine supplementation or a GS gene introduced by an expression vector is essential for cell survival of GS gene knockout cells. In a preferred embodiment the mammalian host cell is a CHO-K1 cell, more preferably a CHO-K1-GS (GS−/−) cell.

Preferably, CHO cells that allow for efficient cell line development processes are metabolically engineered, such as by endogenous glutamine synthetase (GS) knockout to facilitate selection with methionine sulfoximine (MSX).

Non-limiting examples of mammalian cells which can be used in the meaning of this invention are also summarized in Table A. However, derivatives/progenies of those cells, other mammalian cells, including but not limited to human, mice, rat, monkey, and rodent cell lines, can also be used in the present invention, particularly for the production of biopharmaceutical proteins.

TABLE A
Exemplary mammalian production cell lines

| Cell line | Order Number |
|---|---|
| NS0 | ECACC No. 85110503 |
| Sp2/0-Ag14 | ATCC CRL-1581 |
| BHK21 | ATCC CCL-10 |
| BHK TK⁻ | ECACC No. 85011423 |
| HaK | ATCC CCL-15 |
| 2254-62.2 (BHK-21 derivative) | ATCC CRL-8544 |
| CHO | ECACC No. 8505302 |
| CHO wild type | ECACC 00102307 |
| CHO-K1 | ATCC CCL-61 |
| CHOZN ® | Merck SAFC |
| | GS −/− and DHFR −/− |
| CHO-DUKX | ATCC CRL-9096 |
| (═CHO duk⁻; CHO/dhfr⁻, CHO-DXB11) | |
| CHO-DUKX 5A-HS-MYC | ATCC CRL-9010 |
| CHO-DG44 | Urlaub G, et al., 1983. Cell. 33: 405-412. |
| CHO Pro-5 | ATCC CRL-1781 |
| CHO-S | Life Technologies A1136401; CHO-S is derived |
| | from CHO as disclosed in Deaven L L and Petersen |
| | D F, 1973, Chromosoma (Berl.) 41: 129-144; and |
| | Lewis N E et al., 2013, Nature Biotechnology 31: |
| | 759-765 |
| V79 | ATCC CCC-93 |
| B14AF28-G3 | ATCC CCL-14 |
| HEK 293 | ATCC CRL-1573 |
| COS-7 | ATCC CRL-1651 |
| U266 | ATCC TIB-196 |
| HuNS1 | ATCC CRL-8644 |
| CHL | ECACC No. 87111906 |
| CAP¹ | Wölfel J, et al., 2011. BMC Proc. 5(Suppl 8): P133. |
| PER.C6 ® | Pau et al., 2001. Vaccines. 19: 2716-2721. |
| H4-II-E | ATCC CRL-1548 |
| | ECACC No. 87031301 |
| | Reuber, 1961. J. Natl. Cancer Inst. 26: 891-899. |
| | Pitot H C, et al., 1964. Natl. Cancer Inst. Monogr. |
| | 13: 229-245. |
| H4-II-E-C3 | ATCC CRL-1600 |
| H4TG | ATCC CRL-1578 |
| H4-II-E | DSM ACC3129 |
| H4-II-Es | DSM ACC3130 |

¹CAP (CEVEC's Amniocyte Production) cells are an immortalized cell line based on primary human amniocytes. They were generated by transfection of these primary cells with a vector containing the functions E1 and pIX of adenovirus 5. CAP cells allow for competitive stable production of recombinant proteins with excellent biologic activity and therapeutic efficacy as a result of authentic human posttranslational modification.

Cells are most preferred, when being established, adapted, and completely cultivated under serum free conditions, and optionally in media, which are free of any protein/peptide of animal origin. Commercially available media such as Ham's F12 (Sigma, Deisenhofen, Germany), RPMI-1640 (Sigma), Dulbecco's Modified Eagle's Medium (DMEM; Sigma), Minimal Essential Medium (MEM; Sigma), Iscove's Modified Dulbecco's Medium (IMDM; Sigma), CD-CHO (Invitrogen, Carlsbad, CA), serum-free CHO Medium (Sigma), and protein-free CHO Medium (Sigma) are exemplary appropriate nutrient solutions. Any of the media may be supplemented as necessary with a variety of compounds, non-limiting examples of which are recombinant hormones and/or other recombinant growth factors (such as insulin, transferrin, epidermal growth factor, insulin like growth factor), salts (such as sodium chloride, calcium, magnesium, phosphate), buffers (such as HEPES), nucleosides (such as adenosine, thymidine), glutamine, glucose or other equivalent energy sources, antibiotics and trace elements. Any other necessary supplements may also be included at appropriate concentrations that would be known to those skilled in the art. For the growth and selection of genetically modified cells expressing a selectable gene a suitable selection agent is added to the culture medium.

The protein of interest encoded by the expression vector or produced by the methods of the invention is preferably produced in CHO cells in cell culture. Following expression, the recombinant protein is harvested and further purified. The antibody may be recovered from the culture medium as a secreted protein in the harvested cell culture fluid (HCCF) or from a cell lysate (i.e., the fluid containing the content of a cell lysed by any means, including without being limited thereto enzymatic, chemical, osmotic, mechanical and/or physical disruption of the cell membrane and optionally cell wall) and purified using techniques described herein. According to the invention the method comprises providing a harvested cell culture fluid comprising a protein of interest, such as an antibody as starting material, wherein the HCCF is from CHO cell culture. Preferably the protein of interest, such as the antibody, is recovered from the harvested cell culture fluid following cell separation, such as by filtration and/or centrifugation. Thus, in certain embodiments the harvest includes centrifugation and/or filtration to produce a harvested cell culture fluid.

The modified mammalian glutamine synthetase of the first aspect, the expression vector of the second aspect, the nucleic acid if the fifth aspect and the eukaryotic host cell of the sixth aspect may be used in the methods of the present invention. Thus, the embodiments and examples specified with regard to these aspects similarly apply to the aspects relating to methods.

In view of the above, it will be appreciated that the invention also encompasses the following items:
Item 1 provides a modified mammalian glutamine synthetase comprising a mutation at amino acid position 10 and/or 298 in a mammalian glutamine synthetase having an amino acid sequence of SEQ ID NO: 1, 2, 3 or 4 or an amino acid sequence having at least 95% sequence identity with SEQ ID NO: 1, 2, 3 or 4, wherein the mutation is selected from the group consisting of R298K, N10S, N10T and N10Q.
Item 2 specifies the modified mammalian glutamine synthetase of item 2, wherein the mutation does not interfere with substrate-binding.
Item 3 specifies the modified mammalian glutamine synthetase of item 1 or 2, wherein the mutation is R298K and/or N10S, or N10T or N10Q.
Item 4 specifies the modified mammalian glutamine synthetase of any one of items 1 to 3, having an amino acid sequence of SEQ ID NO: 5, 6, 7, 8, 9, 10 or 11.
Item 5 specifies the modified mammalian glutamine synthetase of any one of items 1 to 4, wherein the modified mammalian glutamine synthetase comprising a mutation selected from the group consisting of R298K, N10S, N10T and N10Q (a) has diminished enzymatic activity compared to the same mammalian glutamine synthetase not comprising the mutation; and/or (b) mediates increased selection stringency and/or genetic stability upon transfection with a polynucleotide encoding the modified mammalian glutamine synthetase and a transgene compared to the same mammalian glutamine synthetase not comprising the mutation.
Item 6 specifies the modified mammalian glutamine synthetase of any one of items 1 to 4, wherein the modified mammalian glutamine synthetase comprising a mutation selected from the group consisting of R298K, N10S, N10T and N10Q (a) has diminished enzymatic activity compared to a mammalian glutamine synthetase having the amino acid sequence of SEQ ID NO: 1; and/or (b) mediates increased selection stringency and/or genetic stability upon transfection with a polynucleotide encoding the modified mammalian glutamine synthetase and a transgene compared to a mammalian glutamine synthetase having the amino acid sequence of SEQ ID NO: 1.
Item 7 provides an expression vector comprising a polynucleotide encoding the modified mammalian glutamine synthetase of any one of items 1 to 6.
Item 8 specifies the expression vector of item 7, wherein the expression vector comprises an expression cassette comprising a polynucleotide encoding the modified mammalian glutamine synthetase, preferably wherein the polynucleotide encoding the modified mammalian glutamine synthetase is operably linked to a promoter.
Item 9 specifies the expression vector according to item 7 or 8, wherein the expression vector further comprises at least one polynucleotide encoding a protein of interest and/or a non-coding RNA, preferably an expression cassette comprising at least one polynucleotide encoding a protein of interest and/or a non-coding RNA.
Item 10 specifies the expression vector according to item 9, wherein the protein of interest is a therapeutic protein, preferably selected from the group consisting of a cytokine, a hormone, a fusion protein, an antibody, an antibody-derived molecule and an antibody mimetic.
Item 11 specifies the expression vector according to item 10, wherein the protein of interest is an antibody, preferably wherein the expression vector comprises a polynucleotide comprising a coding sequence for a variable region of the heavy chain and/or a coding sequence for a variable region of the light chain of the antibody.
Item 12 specifies the expression vector according to any one of items 9 to 11, wherein the expression vector comprises a multicistronic expression cassette and/or multiple expression cassettes, preferably wherein the expression vector comprises multiple expression cassettes.
Item 13 specifies the expression vector of any one of items 7 to 12, wherein the expression vector is for stable transfection and the integrating part of the vector comprises the polynucleotide encoding the modified mammalian glutamine synthetase and at least one polynucleotide encoding the protein of interest and/or the non-coding RNA.
Item 14 provides a nucleic acid sequence comprising a polynucleotide encoding the modified mammalian glutamine synthetase of any one of items 1 to 6 operably linked to a mammalian promoter, optionally further comprising at least one polynucleotide encoding a protein of interest and/or a non-coding RNA.
Item 15 provides a eukaryotic host cell comprising a polynucleotide sequence encoding the modified mammalian glutamine synthetase of any one of items 1 to 6, the expression vector of any one of items 7 to 13 or the nucleic acid sequence of item 14.
Item 16 specifies the eukaryotic host cell of item 15, wherein the eukaryotic host cell is (a) a mammalian cell, preferably a rodent cell, more preferably a CHO cell; and/or (b) a GS gene knockout cellt.
Item 17 provides a method for preparing a cell stably expressing a protein of interest and/or a non-coding RNA, comprising (a) introducing the expression vector according to any one of items 7 to 13 or the nucleic acid of item 14, optionally further comprising at least one polynucleotide encoding a protein of interest and/or a non-coding RNA, into a eukaryotic host cell, preferably into a mammalian host cell, more preferably into a CHO cell; and (b) culturing the eukaryotic host cell in a medium comprising no glutamine under conditions to select for the modified mammalian glutamine synthetase, wherein the optional at least one polynucleotide encoding a protein of interest and/or a non-coding RNA is co-integrated with the polynucleotide encoding the modified mammalian glutamine synthetase into the host cell genome.
Item 18 specifies the method of item 17 further comprising (d) a step of isolating a single clone for clonal expansion to prepare a monoclonal cell line.
Item 19 provides a method of producing a protein of interest, comprising (a) introducing the expression vector comprising a polynucleotide encoding a modified mammalian glutamine synthetase according to any one of items 7 to 13 or the nucleic acid of item 14, comprising a mutation selected from the group consisting of R298K, N10S, N10T and N10Q and further comprising at least one polynucleotide encoding a protein of interest into a eukaryotic host cell, preferably into a mammalian host cell, more preferably into a CHO cell; (b) culturing the eukaryotic host cell in a medium comprising no glutamine under conditions to select for the modified mammalian glutamine synthetase, wherein the at least one polynucleotide encoding a protein of interest is co-integrated with the polynucleotide encoding the modified mammalian glutamine synthetase into the host cell genome; (c) optionally isolating single clones for clonal expansion to prepare a monoclonal cell line; (d) culturing the eukaryotic host cell under conditions to produce the protein of interest; and (e) harvesting and optionally purifying the protein of interest.
Item 20 specifies the method of any one of items 17 to 19, wherein the expression vector is introduced by transfection or transduction.
Item 21 specifies the method of item 20, wherein the expression vector is introduced by stable transfection.
Item 22 provides a method of producing a protein of interest, comprising (a) providing the eukaryotic host cell of item 15 or 16 comprising a polynucleotide encoding a mammalian glutamine synthetase comprising a mutation at amino acid position 10 and/or 298 in a mammalian glutamine synthetase having the amino acid sequence of SEQ ID NO: 1, 2, 3 or 4 or an amino acid sequence having at least 95% sequence identity with SEQ ID NO: 1, 2, 3 or 4, wherein the mutation is selected from the group consisting of R298K, N10S, N10T and N10Q and at least one polynucleotide encoding a protein of interest; (b) culturing the eukaryotic host cell under conditions to produce the protein of interest; and (c) harvesting and optionally purifying the protein of interest.
Item 23 specifies the method of any one of items 17 to 22, wherein (a) the eukaryotic host cell is a GS gene knockout cell (GS−/− or GS−/+), and/or (b) culturing the eukaryotic host cell in a medium comprising no glutamine under conditions to select for the modified mammalian glutamine synthetase in step (b) comprises the addition of the GS inhibitor methionine sulfoximine (MSX).
Item 24 specifies the method of any one of items 17 to 23, wherein the eukaryotic host cell is a mammalian cell, preferably a rodent cell, more preferably a CHO cell.
Item 25 specifies the method of any one of items 17 to 24, wherein the cell or monoclonal cell line is generated with increased selection stringency and/or has increased genetic stability and/or has higher productivity compared to a cell or monoclonal cell line generated with the same mammalian glutamine synthetase not comprising the at least one mutation and/or compared to a cell or monoclonal cell line generated with a mammalian glutamine synthetase having the amino acid sequence of SEQ ID NO: 1.
Item 26 provides a kit comprising the expression vector of any one of items 7 to 13 and a cell culture medium not comprising glutamine.
Item 27 provides use of the modified mammalian glutamine synthetase according to items 1 to 6 as a selection marker for monoclonal eukaryotic cell line generation, preferably a mammalian cell line generation, more preferably a rodent cell line generation, even more preferably a CHO cell line generation.
Item 28 provides use of the vector according to any one of items 7 to 13 for producing a protein of interest in a eukaryotic host cell, preferably a mammalian cell, more preferably rodent cell, even more preferably a CHO cell.

### Examples

### Linearization of Plasmids for Transfection

The plasmids used for stable transfection comprises an CMV driven antibody transcription cassette, an ampicillin transgene and a glutamine synthetase transgene as metabolic selection marker. The plasmids were linearized for transfection using Pvul (single cut in the ampicillin transgene). The restriction digest of 20 μg plasmid DNA was conducted at 37° C. for 3 h with Pvul (NEB) according to the manufacturers protocol. The linearized plasmid DNA was purified (Qiagen Plasmid Maxi Kit) according to the manufacturers protocol. The final DNA concentration was determined via Nanodrop spectral photometer.

### Host Cell Cultivation

CHO-K1-GS knockout cell line (also referred to as CHO-K1-GS KO or simply CHO-K1-GS host cell, harboring a genomic knockout of the endogenous gluthamine synthetase gene) was cultivated in host cell medium with added L-glutamine. The cultivation of the host cell was started with a seeding density of 3×10E05 cells per mL. The growth conditions were set to 36.5° C. and 5% CO₂ in a shaking incubator with 120 rpm in shake flasks. Determination of cell density and viability took place in the Cedex HiRes© cell count analyzer.

### Transfection of Linearized Plasmids in CHO

One day before transfection, the host cells were seeded with a cell density of 0.8×10E06 cells/mL in shake flasks. On the day of transfection cell density and viability were determined and the required amount of cells for transfection was centrifuged for 7 minutes with 750 x G. The supernatant was discarded. 10 μg of linearized plasmid DNA per transfection was transfected using the Neon© transfection system (Invitrogen) and electroporation. The electroporation cuvette was filled with 3 mL of electroporation buffer E2, resuspended cells (5×10E6) in 89 μl of buffer R and mixed with 10 μg of linearized plasmid. The transfection was performed using 1500 Volt, 10 mS and a pulse of 2. Transfected cells were transferred in 5 ml prewarmed host cell medium in T25 ml flasks and incubated with 8% CO₂ and 37° C. for at least 24 h.

### Selection of Stable CHO Pools

24 h after transfection, cells were transferred into selection medium (medium without L-glutamine). 10 mL of selection medium was prewarmed for every pool in T75 Flasks. Two stable pools were cultivated for each transfection. Cells were centrifuged (for 7 minutes with 750 x G), resuspended in 20 mL of selection medium and incubated with 8% CO₂ at 37° C. During selection, cells were monitored by microscopy. Additional selection medium (5 mL) was added after 7 days.

### Passaging and Production Run of Stable Pools

Once cells reached viability of at least 70% and a doubling time of at least 48 hours, selection phase was considered successful. After selection, cells were passaged every two to three days starting with 3×10E05 cells per mL in 30 mL total volume of selection medium (not containing L-glutamine) in 125 mL shake flasks at 36.5° C. and 5% CO₂ at 120 rpm shaking. Samples for titer measurements were taken regularly. If antibody titer was stable for at least 14 days, indicating phenotypic stability, an at least 7 days production run was started.

For the production run cells were seeded with a density of 7×10E05 viable cells per mL in 30 mL total volume basal medium without glutamine in shake flasks and cells were cultivated at 34.5° C., 5% CO₂ and 120 rpm shaking. During the 7 day production run, cells were counted and samples for pH, glucose and titer measurement were collected daily to adjust pH and glucose feeding if necessary. Starting from day 2 a daily feed (w/o glutamine) was supplemented. Parameters, such as pH was determined using the RAPIDlab 348Ex© and glucose was measured using an EKF diagnostics device. At day 7, cell culture medium supernatant was analyzed following centrifugation for antibody titers using a ForteBio Octet© device with protein A Biosensors. Dilution of samples and standard curve were processed in the same production medium.

### Example 1: Antibody Production in CHO Cells Using CHO Wildtype GS as Metabolic Selection Marker

CHO-K1-GS cells once transfected with a vector carrying a glutamine synthetase from Cricetulus griseus (CHO wildtype glutamine synthetase (GS), SEQ ID NO: 1) selection marker can survive selection under cultivation in medium without supplementation of L-glutamine, when the transgene vector is stably integrated into the genome of the cell.

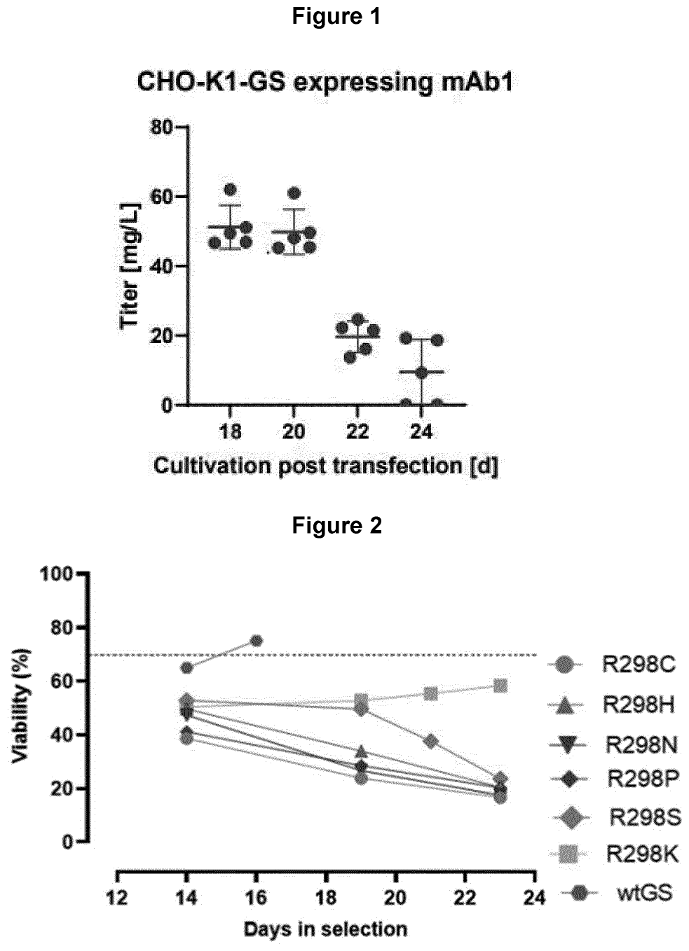

CHO-K1-GS cells were transfected with a vector carrying the expression cassette of a monoclonal antibody 1 (mAB1) and a CHO wildtype GS. Following selection, stable CHO pools were passaged as described above and samples for titer measurements of mAb1 were taken regularly. Under the applied experimental conditions, the stable CHO pools remained stable and productive for up to ˜20 days post transfection with decreasing levels of productivity (titer) thereafter (FIG. 1).

### Example 2: Antibody Production in CHO Cells Using CHO GS with Mutations at Position R298 as Selection Marker

To functionally test novel CHO-based glutamine synthetase variants, selection stringency (duration to reach>70% viability after transfection), amount of productive passages and specific productivity was measured over time. For CHO pools, which remain productive after prolonged passaging, a 7-day production run in shaking flasks was subsequently conducted in a controlled process set-up to additionally evaluate cell culture process parameters.

CHO-K1-GS cells were transfected with a vector carrying the expression cassette of mAb1 and the CHO GS harboring various mutations at amino acid position 298 (R298 of SEQ ID NO: 1). Viable cell density (VCD), viability and productivity were measured over time during selection phase. From all tested point mutations, only CHO GS R298K conferred survival during selection phase suggesting that R298 is a conserved amino acid residue. Cells transfected with CHO GS R298K mutation took longer to fully recover from selection (FIG. 2) and cell growth was slower in comparison to the CHO wildtype GS (wtGS), suggesting that selection stringency is increased.

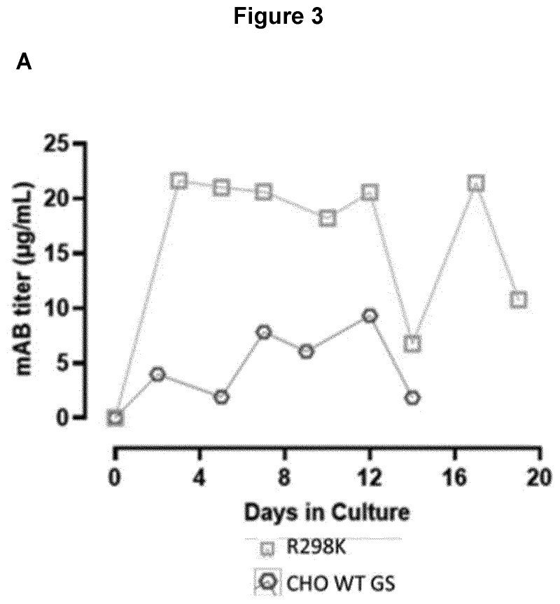

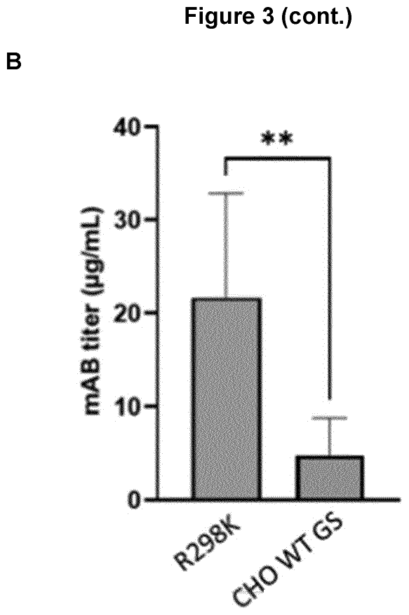

After the transfected cells fully recovered from selection and reached at least 70% and a doubling time of at least 48 hours, the cells were transferred to shake flasks for passaging and samples for titer measurement of mAb1 were taken regularly. CHO GS R298K additionally conferred significantly higher productivity and cell pools remained more stable over the course of 20 days compared to wildtype CHO GS (CHO WT GS) (FIG. 3A) and the mean productivity of CHO GS R298K was about 4-fold increased compared to CHO wildtype GS (FIG. 3B).

Overall, CHO cells transfected with the CHO GS variant R298K showed an increased selection stringency and significantly higher productivity. Moreover, cell pools show an increased phenotypic stability and remain productive for at least 45 days (data not shown), whereas cells transfected with CHO WT GS completely lost their productivity after ˜20 days post transfection.

### Example 3: Antibody Production in CHO Cells Using CHO GS with Mutations at Position N10 as Selection Marker

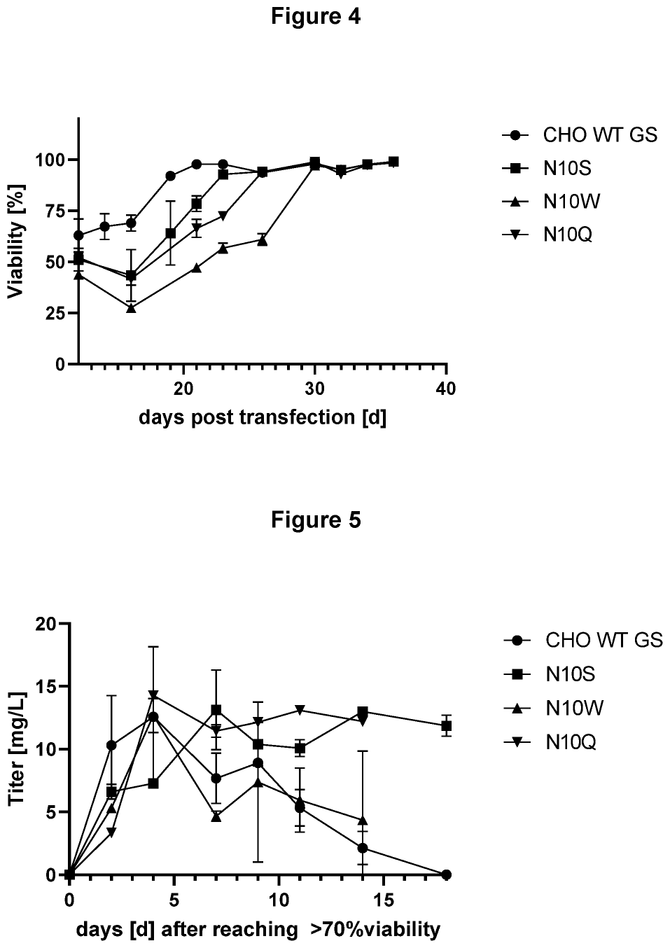

CHO-K1-GS cells were transfected with a vector carrying the expression cassette of mAb1 and the glutamine synthetase from Cricetulus griseus harboring various mutations at amino acid position N10 (of SEQ ID NO: 1). Viable cell density (VCD), viability and productivity were measured over time during selection phase. Cells transfected with the CHO GS carrying a N10S, N10Q and N10W mutation took longer to fully recover from selection (FIG. 4) and cell growth was slower in comparison to the CHO WT GS, suggesting that the selection stringency is increased.

All tested variants (N10S, N10Q and N10W) showed similar levels of productivity initially compared to wildtype CHO GS (CHO WT GS). Unexpectedly, CHO GS N10S and N10Q showed a significant increase in phenotypic stability compared to CHO WT GS and N10W, both showing a decline in productivity after 4 days in selection (FIG. 5). N10S and N10Q remained stable for an additional production run in shaking flasks (data not shown).

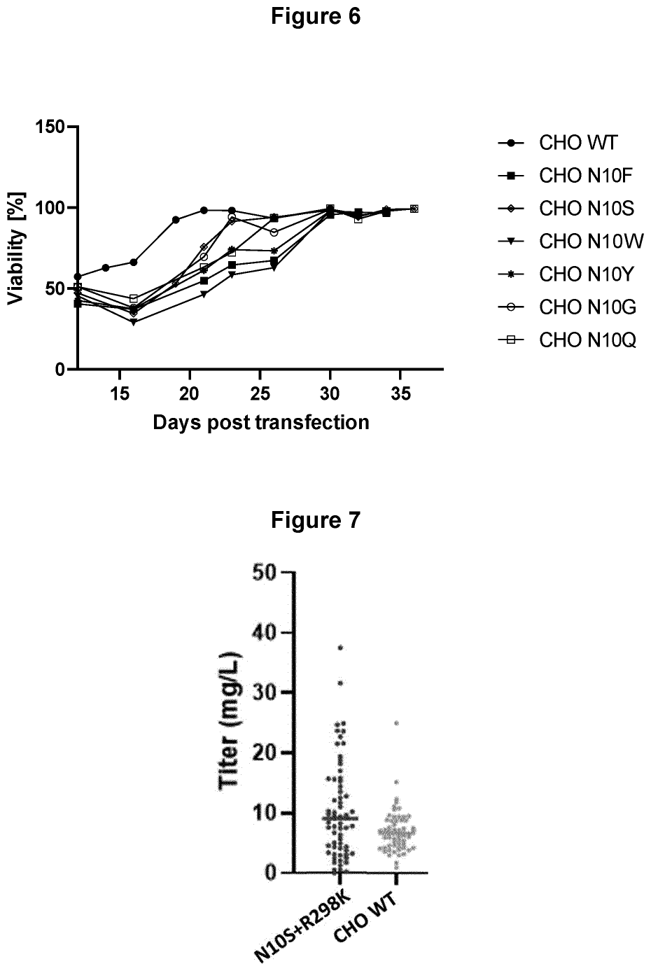

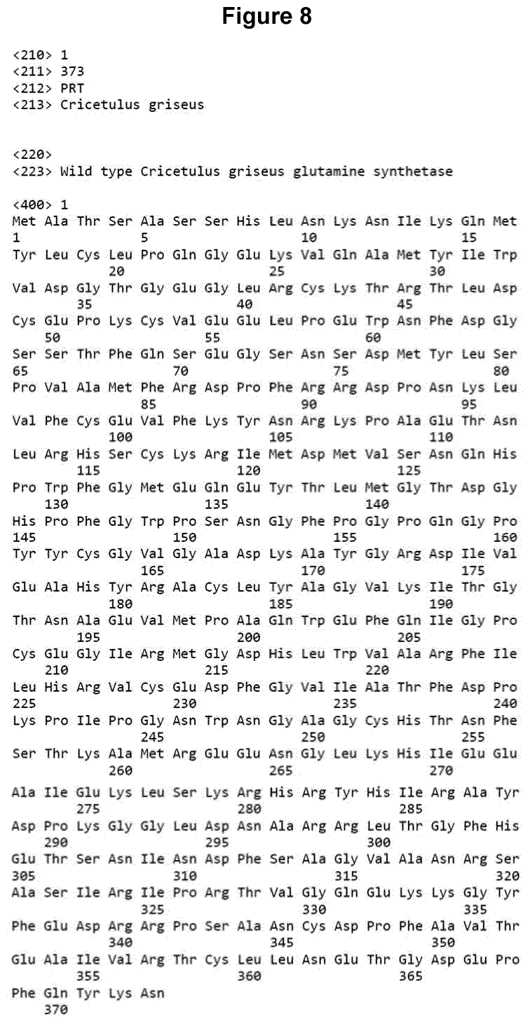

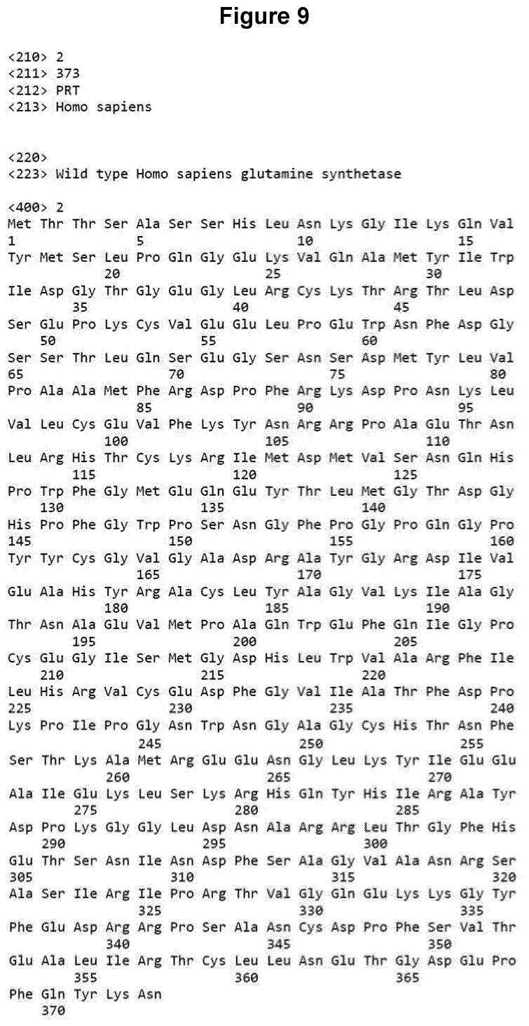

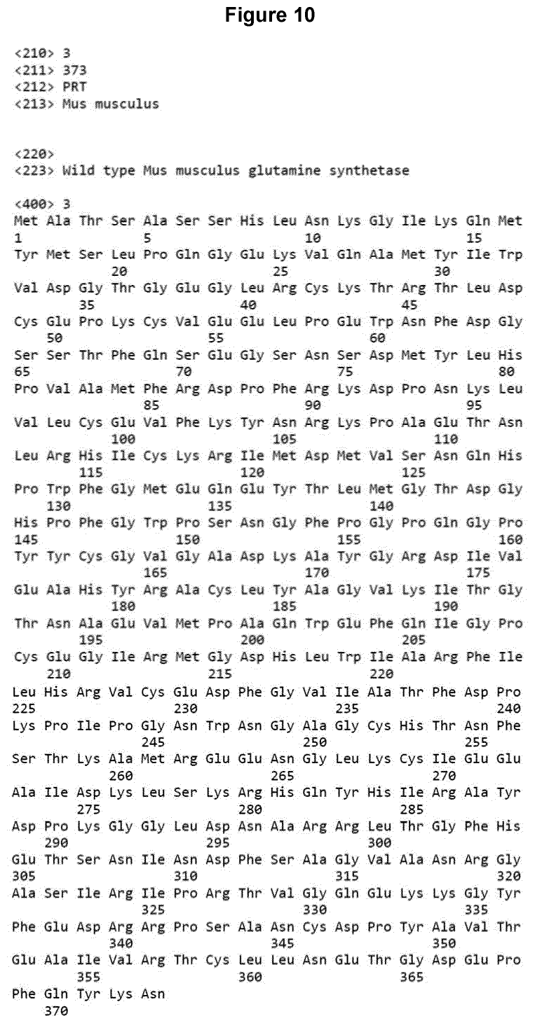

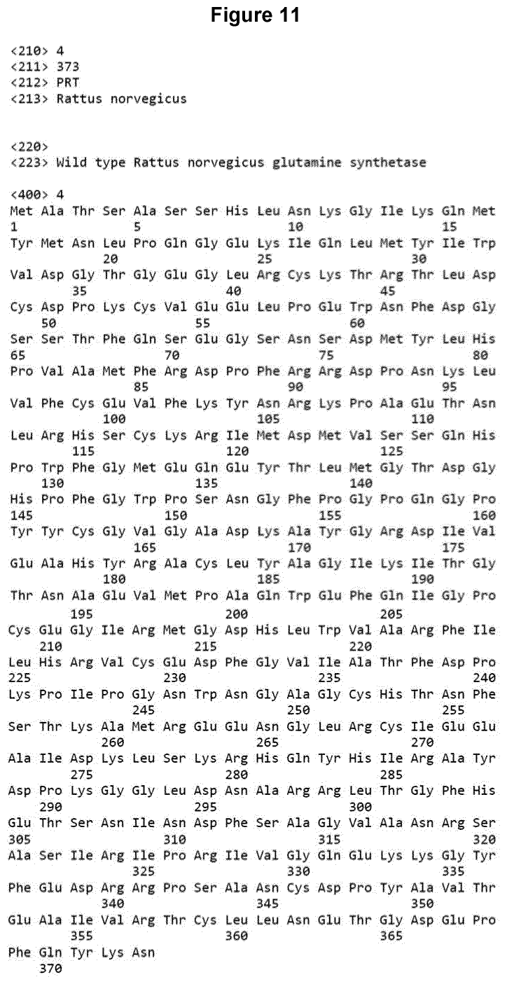

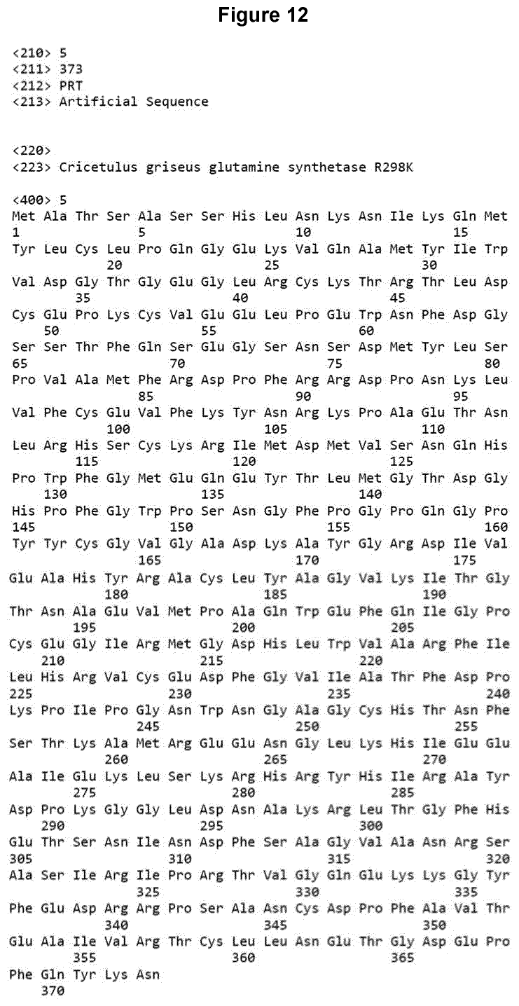

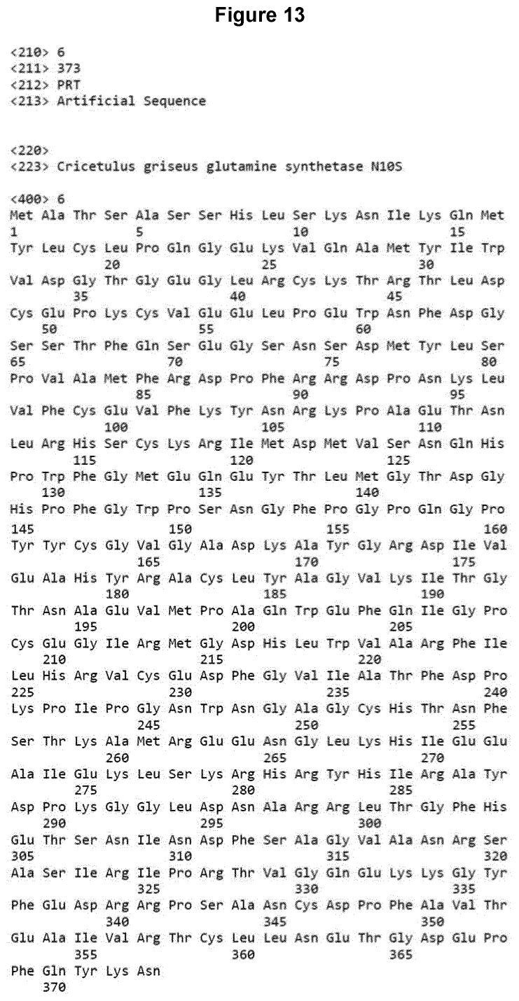

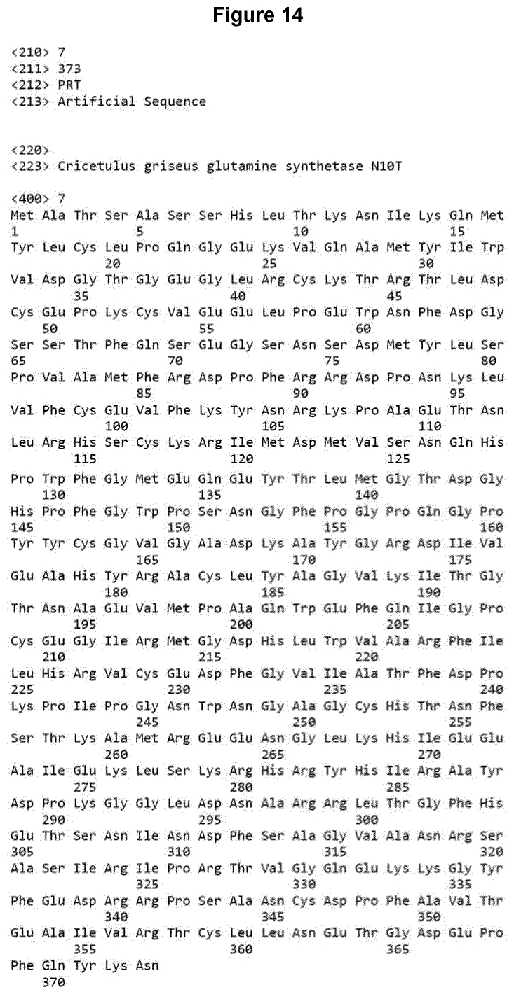

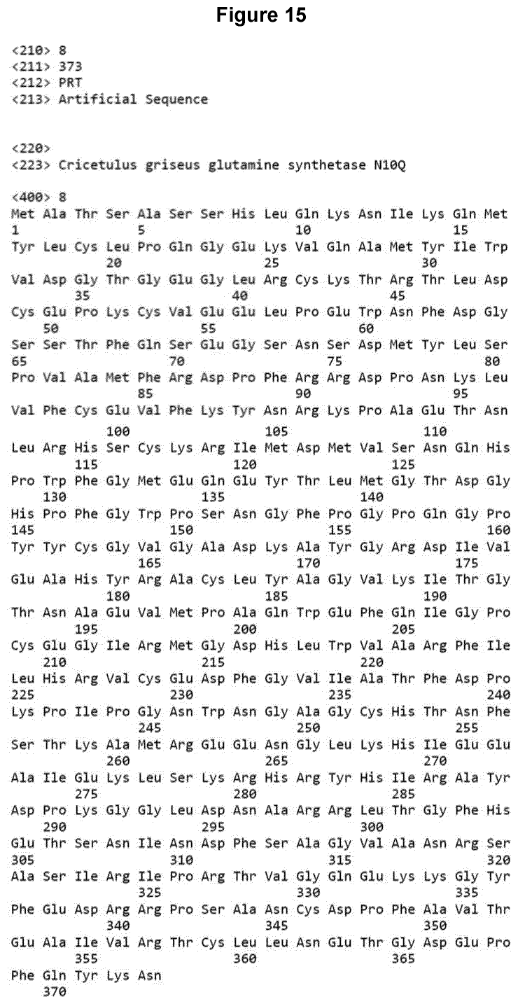

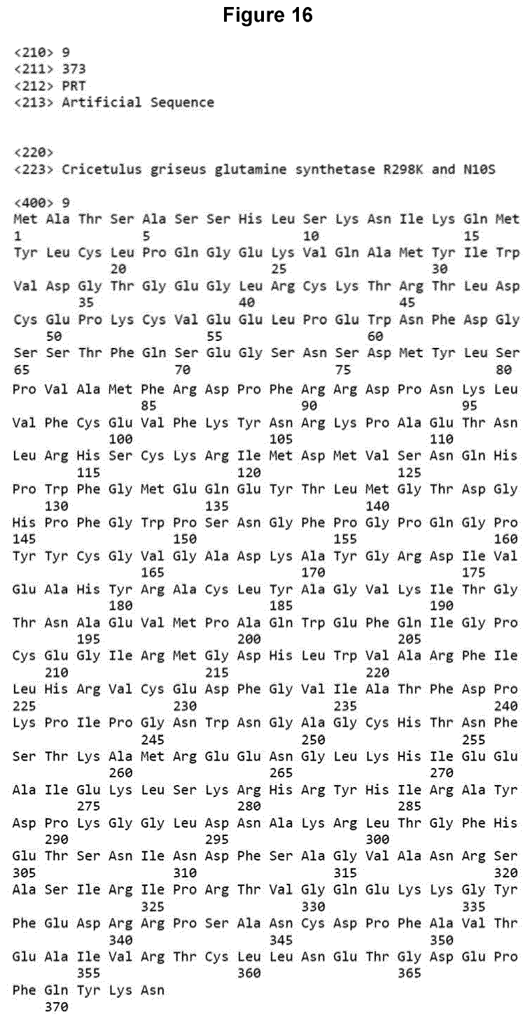

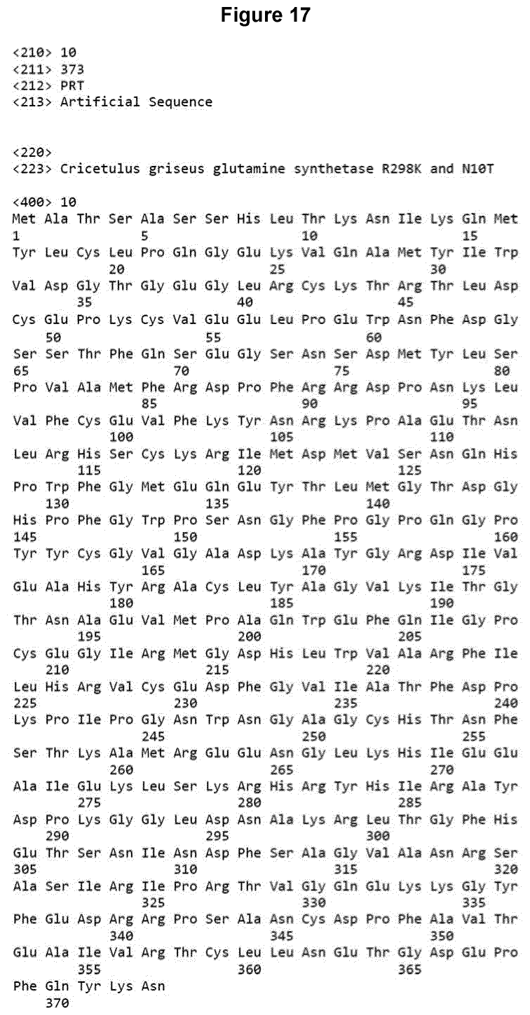

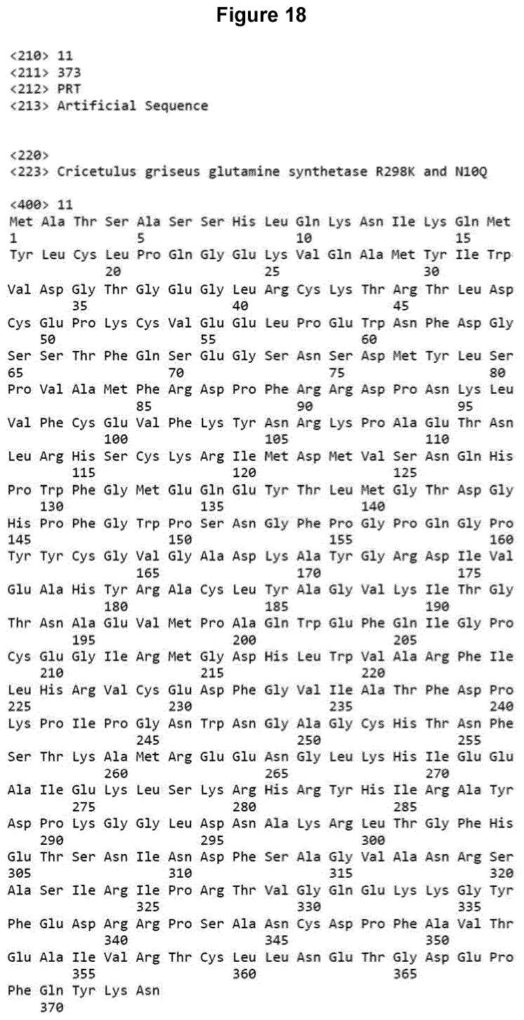

In addition, CHO GS N10F, N10Y and N10G have been tested alongside with CHO N10Q, N10S and N10W in addition to wildtype CHO GS. All variants were functional and resulted in stable pool generation although with varying selection stringency (outgrowth phase after transfection varies strongly) (FIG. 6). However, except for CHO GS N10S and CHO GS N10Q none of the tested variants showed phenotypic stability (data not shown). Although not tested CHO GS N10T is expected to show a similar phenotype as CHO GS N10S due to the structural similarity of the amino acids Serine and Tyrosine.

Overall, CHO cells transfected with the CHO GS variants N10S and N10Q show an increased selection stringency and significantly longer phenotypic stability compared to the wildtype selection marker.

### Example 4: Antibody Production in CHO Cells Using CHO GS with Combined Mutations N10S and R298K as Selection Marker

Examples 2 and 3 demonstrated that specific mutations in R298 and N10 conferred increased selection stringency and significantly longer phenotypic stability compared to the wildtype GS selection marker. Further, CHO GS with combined mutations in N10 and R298 were tested. Specifically, mutations N10S and R298K were combined and tested for stable pool generation as well as productivity assessment on clonal level at 384-screening stage.

CHO-K1-GS cells were transfected with a vector carrying the expression cassette of mAb1 and the glutamine synthetase from Cricetulus griseus harboring mutations N10S and R298K. The double mutant was found to generate stable cell pools with increased productivity (FIG. 7). Particularly the genomic stability was clearly superior compared to CHO WT (data not shown).

### SEQUENCE LISTING

| SEQ ID NO: | description | FIG. |
|---|---|---|
| 1 | Wild type Cricetulus griseus glutamine synthetase | 8 |
| 2 | Wild type Homo sapiens glutamine synthetase | 9 |
| 3 | Wild type Mus musculus glutamine synthetase | 10 |
| 4 | Wild type Rattus norvegicus glutamine synthetase | 11 |
| 5 | Cricetulus griseus glutamine synthetase R298K | 12 |
| 6 | Cricetulus griseus glutamine synthetase N10S | 13 |
| 7 | Cricetulus griseus glutamine synthetase N10T | 14 |
| 8 | Cricetulus griseus glutamine synthetase N10Q | 15 |
| 9 | Cricetulus griseus glutamine synthetase R298K and N10S | 16 |
| 10 | Cricetulus griseus glutamine synthetase R298K and N10T | 17 |
| 11 | Cricetulus griseus glutamine synthetase R298K and N10Q | 18 |

1. A modified mammalian glutamine synthetase comprising a mutation at amino acid position 10 and/or 298 in a mammalian glutamine synthetase having an amino acid sequence of SEQ ID NO: 1, 2, 3 or 4 or an amino acid sequence having at least 95% sequence identity with SEQ ID NO: 1, 2, 3 or 4, wherein the mutation is selected from the group consisting of R298K, N10S, NOT and N10Q.

2. The modified mammalian glutamine synthetase of claim 2, wherein the mutation does not interfere with substrate-binding.

3. The modified mammalian glutamine synthetase of claim 1, wherein the modified mammalian glutamine synthetase comprising a mutation selected from the group consisting of R298K, N10S, N10T and N10Q (a) has diminished enzymatic activity compared to the same mammalian glutamine synthetase not comprising the mutation; and/or (b) mediates increased selection stringency and/or genetic stability upon transfection with a polynucleotide encoding the modified mammalian glutamine synthetase and a transgene compared to the same mammalian glutamine synthetase not comprising the mutation.

4. An expression vector comprising a polynucleotide encoding the modified mammalian glutamine synthetase of claim 1.

5. The expression vector according to claim 4, wherein the expression vector further comprises at least one polynucleotide encoding a protein of interest and/or a non-coding RNA.

6. A nucleic acid sequence comprising a polynucleotide encoding the modified mammalian glutamine synthetase of claim 1 operably linked to a eukaryotic promoter, optionally further comprising at least one polynucleotide encoding a protein of interest and/or a non-coding RNA.

7. A eukaryotic host cell comprising a polynucleotide sequence encoding the modified mammalian glutamine synthetase of claim 1.

8. A method for preparing a cell stably expressing a protein of interest and/or a non-coding RNA, comprising
(a) introducing the expression vector according to claim 4, optionally further comprising at least one polynucleotide encoding a protein of interest and/or a non-coding RNA, into a eukaryotic host cell; and
(b) culturing the eukaryotic host cell in a medium comprising no glutamine under conditions to select for the modified mammalian glutamine synthetase, wherein the optional at least one polynucleotide encoding a protein of interest and/or a non-coding RNA is co-integrated with the polynucleotide encoding the modified mammalian glutamine synthetase into the host cell genome.

9. A method of producing a protein of interest, comprising (a) introducing the expression vector comprising a polynucleotide encoding a modified mammalian glutamine synthetase according to claim 4, comprising a mutation selected from the group consisting of R298K, N10S, N10T and N10Q and further comprising at least one polynucleotide encoding a protein of interest into a eukaryotic host cell;
(b) culturing the eukaryotic host cell in a medium comprising no glutamine under conditions to select for the modified mammalian glutamine synthetase, wherein the at least one polynucleotide encoding a protein of interest is co-integrated with the polynucleotide encoding the modified mammalian glutamine synthetase into the host cell genome;
(c) optionally isolating single clones for clonal expansion to prepare a monoclonal cell line;
(d) culturing the eukaryotic host cell under conditions to produce the protein of interest; and
(e) harvesting and optionally purifying the protein of interest.

10. (canceled)

11. A method of producing a protein of interest, comprising
(a) providing the eukaryotic host cell of claim 7 comprising a polynucleotide encoding a mammalian glutamine synthetase comprising a mutation at amino acid position 10 and/or 298 in a mammalian glutamine synthetase having the amino acid sequence of SEQ ID NO: 1, 2, 3 or 4 or an amino acid sequence having at least 95% sequence identity with SEQ ID NO: 1, 2, 3 or 4, wherein the mutation is selected from the group consisting of R298K, N10S, N10T and N10Q and at least one polynucleotide encoding a protein of interest;
(b) culturing the eukaryotic host cell under conditions to produce the protein of interest; and
(c) harvesting and optionally purifying the protein of interest.

12. The eukaryotic host cell of claim 7, wherein the eukaryotic host cell is (a) a mammalian host cell and/or (b) a GS knockout mutant.

13. A kit comprising the expression vector of claim 4 or 5 and a cell culture medium not comprising glutamine.

14-15. (canceled)

16. A eukaryotic host cell comprising the expression vector of claim 4.

17. A eukaryotic host cell comprising the nucleic acid sequence of claim 6.

18. A method for preparing a cell stably expressing a protein of interest and/or a non-coding RNA, comprising
(a) introducing the nucleic acid of claim 6, optionally further comprising at least one polynucleotide encoding a protein of interest and/or a non-coding RNA, into a eukaryotic host cell; and
(b) culturing the eukaryotic host cell in a medium comprising no glutamine under conditions to select for the modified mammalian glutamine synthetase, wherein the optional at least one polynucleotide encoding a protein of interest and/or a non-coding RNA is co-integrated with the polynucleotide encoding the modified mammalian glutamine synthetase into the host cell genome.

19. A method of producing a protein of interest, comprising
(a) introducing the nucleic acid of claim 6, comprising a mutation selected from the group consisting of R298K, N10S, N10T and N10Q and further comprising at least one polynucleotide encoding a protein of interest into a eukaryotic host cell;
(b) culturing the eukaryotic host cell in a medium comprising no glutamine under conditions to select for the modified mammalian glutamine synthetase, wherein the at least one polynucleotide encoding a protein of interest is co-integrated with the polynucleotide encoding the modified mammalian glutamine synthetase into the host cell genome;
(c) optionally isolating single clones for clonal expansion to prepare a monoclonal cell line;
(d) culturing the eukaryotic host cell under conditions to produce the protein of interest; and
(e) harvesting and optionally purifying the protein of interest.

20. A method of producing a protein of interest, comprising
(a) providing the eukaryotic host cell of claim 16 comprising a polynucleotide encoding a mammalian glutamine synthetase comprising a mutation at amino acid position 10 and/or 298 in a mammalian glutamine synthetase having the amino acid sequence of SEQ ID NO: 1, 2, 3 or 4 or an amino acid sequence having at least 95% sequence identity with SEQ ID NO: 1, 2, 3 or 4, wherein the mutation is selected from the group consisting of R298K, N10S, N10T and N10Q and at least one polynucleotide encoding a protein of interest;
(b) culturing the eukaryotic host cell under conditions to produce the protein of interest; and
(c) harvesting and optionally purifying the protein of interest.

21. A method of producing a protein of interest, comprising
(a) providing the eukaryotic host cell of claim 17 comprising a polynucleotide encoding a mammalian glutamine synthetase comprising a mutation at amino acid position 10 and/or 298 in a mammalian glutamine synthetase having the amino acid sequence of SEQ ID NO: 1, 2, 3 or 4 or an amino acid sequence having at least 95% sequence identity with SEQ ID NO: 1, 2, 3 or 4, wherein the mutation is selected from the group consisting of R298K, N10S, N10T and N10Q and at least one polynucleotide encoding a protein of interest;
(b) culturing the eukaryotic host cell under conditions to produce the protein of interest; and
(c) harvesting and optionally purifying the protein of interest.

22. The method of claim 8, wherein the eukaryotic host cell is (a) a mammalian host cell and/or (b) a GS knockout mutant.

23. The method of claim 9, wherein the eukaryotic host cell is (a) a mammalian host cell and/or (b) a GS knockout mutant.

24. The method of claim 10, wherein the eukaryotic host cell is (a) a mammalian host cell and/or (b) a GS knockout mutant.

25. The method of claim 11, wherein the eukaryotic host cell is (a) a mammalian host cell and/or (b) a GS knockout mutant.
